(19) United States
(12) Patent Application Publication
(10) Pub. No.: US 2005/0266068 A1
(43) Pub. Date: Dec. 1, 2005

(54) Cardiolipin molecules and methods of synthesis

(75) Inventors: Moghis U. Ahmad, Wadsworth, IL (US); Murali K. Ukkalam, Waukegan, IL (US); Imran Ahmad, Wadsworth, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Neopharm, Inc., Lake Forrest, IL (US)

(21) Appl. No.: 11/105,970

(22) Filed: Apr. 14, 2005

Related U.S. Application Data
(63) Continuation of application No. PCT/US03/27806, filed on Sep. 5, 2003.
(60) Provisional application No. 60/429,285, filed on Nov. 26, 2002.

(51) Int. Cl. A61K 48/00; C07F 5/06; C07K 14/47; A61K 9/127; A61K 38/16

(52) U.S. Cl. 424/450; 554/76; 530/352; 536/109; 514/44

(57) ABSTRACT

The invention provides new synthetic routes for cardiolipin with different fatty acids and/or alkyl chains with varying chain length and also with or without unsaturation, particularly a short-chain cardiolipin. The methods comprise reacting a 1,2-O-sn-diacyl/1,2-O-sn-dialkyl glycerol or a 2-O-protected glycerol, with a phosphoramidite reagent or a phosphate triester to produce a protected cardiolipin, which is deprotected to prepare the short chain cardiolipin. The reaction schemes can be used to generate new variants of cardiolipin. The cardiolipin prepared by the present methods can be incorporated into liposomes, which can also include active agents such as hydrophobic or hydrophilic drugs. Such liposomes can be used to treat diseases or in diagnostic and/or analytical assays. Liposomes can also include ligands for targeting a particular cell type or specific tissue.

### CROSS REFERENCE TO RELATED PATENT APPLICATIONS

This application is a continuation of PCT/US03/27806 filed on Sep. 5, 2003, which claims priority to U.S. Provisional Application No. 60/429,285 filed on Nov. 26, 2002. The disclosures of these applications are incorporated herein in their entireties by reference thereto.

### FIELD OF THE INVENTION

This invention pertains to novel synthetic methods for preparing cardiolipin analogs/variants, and compositions containing them. The invention also pertains to liposome formulations or complexes or emulsions containing active agents or drugs and their use in the treatment of diseases in humans and animals.

### BACKGROUND OF THE INVENTION

Liposomal formulations have the capacity to increase the solubility of hydrophobic drugs in aqueous solution. They often reduce the side effects associated with drug therapy and they provide flexible tools for developing new formulations of active agents.

Liposomes are commonly prepared from natural phospholipids such as phosphatidylcholine, phosphatidylethanolamine, phosphatidylserine, phosphatidylglycerol, phosphatidic acid, and phosphatidylinositol. Anionic phospholipids, such as phosphatidyl glycerol and cardiolipin, can be added to generate a net negative surface charge that provides for colloid stabilization. These components are often purified from natural sources and in some cases they can be chemically synthesized.

The nature and density of the surface charge of liposomes influences stability, kinetics, biodistribution, and interaction with, and uptake by target cells. Liposome surface charge also influences the tendency for liposomes to aggregate, which makes liposomes difficult to work with and affects uptake by target cells. In this respect, liposomes with a neutral surface charge have the highest tendency to aggregate, but are less likely to be cleared by cells of the reticuloendothelial system (RES) after systemic administration. Negatively charged liposomes, on the other hand, exhibit reduced aggregation and increased stability, but exhibit non-specific cellular uptake in vivo. Thus, it has been suggested that a small amount of negatively charged lipids may stabilize neutral liposomes against an aggregation-dependent uptake mechanism (see, e.g., Drummond et al., Pharm. Rev., 51, 691-743 (1999)).

Cardiolipin (also known as diphosphatidyl glycerol) constitutes a class of complex anionic phospholipids that is typically purified from cell membranes of tissues associated with high metabolic activity, including the mitochondria of heart and skeletal muscles. The negative surface charge of cardiolipin, therefore, stabilizes liposomes against aggregation-dependent uptake, as discussed above. In animal tissues and mitochondria, cardiolipin contains up to 90% of linoleic acid (18:2). Yeast cardiolipin differs in having more oleic (18:1) and palmitoleic (16:1) fatty acids, while the bacterial lipid contains saturated and monoenoic fatty acids with 14 to 18 carbons. However cardiolipin having shortchain fatty acids are unknown till now. The potential effects of the length and nature of cardiolipin fatty acid chains (i.e., saturated or unsaturated) on liposome aggregation have not been elucidated.

Methods for synthesizing cardiolipin comprising short fatty acid chains (“short chain cardiolipin”) have not yet been described. Generally, known methodologies for synthesizing cardiolipin are mainly divided in two groups: (a) coupling the primary hydroxyl groups of a 2-protected glycerol with 1,2-diacyl-sn-glycerol using a phosphorylating agent and (b) condensation at both primary hydroxyl groups of a 2-protected glycerol with phosphatidic acid in the presence of 2,4,6-triisopropylbenzenesulfonylchloride (TPS) or pyridine (see, e.g., Ramirez et al., Synthesis, 11, 769-770 (1976), Duralski et al., Tetrahedron Lett. 39, 1607-1610 (1998), Saunders and Schwarz, J. Am. Chem. Soc. 88, 3844-3847 (1966), Mishina et al., Bioorg. Khim. 11, 992-994 (1985), and Stepanov et al., Zh. Org., Khim. 20, 985-988 (1984)). Cardiolipin has also been generated via a reaction between the silver salt of diacylglycerophosphoric acid benzyl ester with 1,3-diiodopropanol benzyl ether or 1,3-diiodopropanol t-butyl ether (see, e.g., De Haas et al., Biochim. Biophys. Acta, 116, 114-124 (1966) and Inoue et al., Chem. Pharm. Bull. 11, 1150-1156 (1963)). Although the schemes were suitable for the preparation of small quantities of cardiolipin, those were unattractive for the routine preparation of larger quantities due to the many steps involved, the requirement for careful purification of intermediates and the use of highly photosensitive silver salt derivatives and unstable iodo intermediates.

Phosphate triesters and phosphoramidite esters have been used extensively in nucleic acid synthesis to form phosphate linkages, and to a lesser extent in phospholipid synthesis (see, e.g., Browne et al., J. Chem. Soc. Perkin Trans, 1, 653-657 (2000)). In this respect, Browne et al., supra, describes the preparation of phospholipid analogs, particularly phosphorylcholine analogs, using phosphoramidite methodologies. The phosphatidylinositols PtdIns(4,5)P₂ and PtdIns(3,4,5)P₃, and derivatives thereof, have been prepared using a variety of phosphoramidite reagents, including N,N-diisopropylphosphoramidite (see, e.g., Watanabe et al., Tetrahedron Lett. 35, 123-124 (1994)), difluorenyl phosphoramidite (see, e.g., Watanabe et al., Tetrahedron Lett. 38, 7407-7410 (1997)), and a reagent produced by reacting a diacylglycerol with (benzyloxy)(N,N-diisopropylamino)chlorophosphine (see, e.g., Chen et al., J. Org. Chem., 61, 6305-6312 (1996) and Prestwich et al., Acc. Chem. Res., 29, 503-513 (1996)). In addition, phosphotriester analogs of PtdIns(4,5)P₂ and PtdIns(3,4,5)P₃ have been prepared utilizing the phosphoramidite reagent 2-cyano-ethyl N,N,N,N-tetraisopropylphosphorodiamidite (see, e.g., Gu et al., J. Org. Chem, 61, 8642-8647 (1996)). Moreover, Murakami et al., J. Org. Chem, 64, 648-651 (1999) describe the synthesis of phosphatidyl glycerol from 2,5-dibenzyl-D-mannitol utilizing methyl tetraisopropylphosphorodiamidite as a phosphorylating agent. The use of phosphate triesters and phosphoramidite esters in preparing phospholipids such as cardiolipin, particularly cardiolipin species having varying fatty acid chain lengths, however, is not well established.

New synthetic methods are needed that can be used to prepare large quantities of saturated and unsaturated cardiolipin species having varying fatty acid chain length, particularly “short chain cardiolipins”. Such methods would increase the availability of a wider variety of cardiolipin species and would diversify the lipids available for development of new liposomal formulations containing active agents, which will have more defined compositions than those currently available.

The invention provides such methods and compositions. These and other advantages of the invention, as well as additional inventive features, will be evident from the description of the invention provided herein.

### BRIEF SUMMARY OF THE INVENTION

The invention provides novel synthetic methodologies for preparing cardiolipin having varying fatty acids and/or alkyl chains with varying length and saturation/unsaturation. The methods comprises of (a) reacting an optically pure 1,2-O-diacyl-sn-glycerol or 1,2-O-dialkyl-sn-glycerol with one or more phosphoramidite reagent(s) or one or more phosphate triester(s), (b) coupling the product of (a) with a 2-protected glycerol, wherein a protected cardiolipin is produced, and (c) deprotecting the protected cardiolipin, such that the cardiolipin is prepared. The invention also provides a method for preparing cardiolipin having varying fatty acid chain lengths comprising (a) reacting a 2-O-protected glycerol with one or more phosphoramidite reagents, wherein a phosphorylating agent is produced, (b) reacting the phosphorylating agent with an optically pure 1,2-O-diacyl-sn-glycerol or 1,2-O-dialkyl-sn-glycerol, wherein a protected cardiolipin is produced, and (c) deprotecting the protected cardiolipin, such that the cardiolipin is prepared.

The cardiolipin prepared by the present methods can be incorporated into liposomes, which can also include active agents such as hydrophobic or hydrophilic drugs, antisense nucleotides or diagnostic agents. Such liposomes can be used to treat diseases or in diagnostic and/or analytical assays.

### BRIEF DESCRIPTION OF THE DRAWINGS

FIG. 1 depicts the general structure of cardiolipin.

FIG. 2 depicts one scheme for synthesizing cardiolipin.

FIG. 3 depicts an alternative synthetic scheme for cardiolipin.

FIG. 4 depicts an alternative synthetic scheme for cardiolipin.

FIG. 5 depicts an alternative synthetic scheme for cardiolipin.

FIG. 6 depicts an alternative synthetic scheme for cardiolipin ether analogs.

FIG. 7 depicts an alternative synthetic scheme for cardiolipin ether analogs.

### DETAILED DESCRIPTION OF THE INVENTION

The present invention describes methods for the synthesis of cardiolipin variants and analogs having the general formulas I, II, and III, as well as compositions containing such variants and analogs. 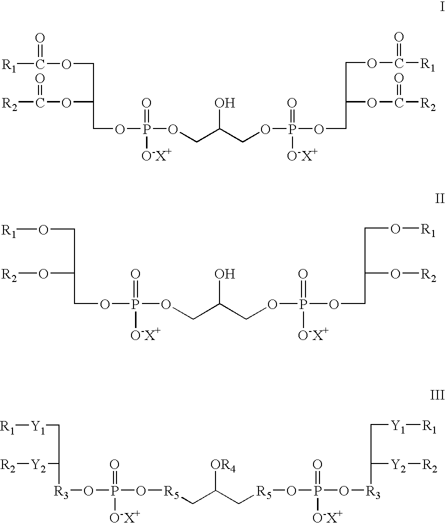
In Formula III, Y₁ and Y₂ are the same or different and are —O—C(O)—, —O—, —S—, —NH—C(O)— or the like. In Formulas I, II and III, R₁ and R₂ are the same or different and are H, saturated and/or unsaturated alkyl group, preferably a C₂ to C₃₄ saturated and/or unsaturated alkyl group. In Formula III, R₃ is (CH₂)ₙ and n=0-15. In Formula III, R₄ is hydrogen, alkyl, substituted alkyl, cycloalkyl, substituted cycloalkyl, a peptide, dipeptide, polypeptide, protein, carbohydrate (such as glucose, mannose, galactose, polysaccharide and the like), heterocyclic, nucleoside, polynucleotide and the like. In Formula III, R₅ is a linker, which may (or may not be) added in the molecule depending on the need and applications. However, where added, R₅ can comprise alkyl, substituted alkyl, cycloalkyl, substituted cycloalkyl, alkoxy, polyalkyloxy (such as pegylated ether of containing from about 1 to 500 alkyloxymers (and can have at least about 10 alkyloxy mers, such as at least about 50 alkyloxy mers or at least about 100 alkyloxy mers, such as at least about 200 alkyloxy mers or at least about 300 alkyloxy mers or at least about 400 alkyloxy mers), substituted polyalkyloxy and the like), a peptide, dipeptide, polypeptide, protein, carbohydrate such as glucose, mannose, galactose, polysaccharides and the like. In Formulas I, II and III, X is hydrogen or a non-toxic cation, preferably ammonium, sodium, potassium, calcium, barium ion and the like.

The term “alkyl” encompasses saturated or unsaturated straight-chain and branched-chain hydrocarbon moieties. The term “substituted alkyl” comprises alkyl groups further bearing one or more substituents selected from hydroxy, alkoxy (of a lower alkyl group), mercapto (of a lower alkyl group), cycloalkyl, substituted cycloalkyl, halogen, cyano, nitro, amino, amido, imino, thio, —C(O)H, acyl, oxyacyl, carboxyl, and the like.

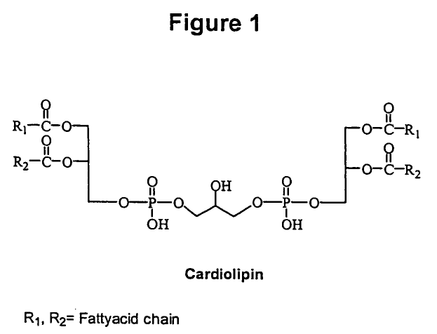

In the most preferred embodiment Y₁ and Y₂ in Formula III are —O—C(O)— or —O—. R₃ is most preferably is CH₂. Also, in Formulas I, II, and III, R₁ and R₂ are the same and are a C₂ to C₁₃ saturated and/or unsaturated alkyl group, more preferably between 4 and 14 carbon atoms (such as between about 6 and 12 carbon atoms). X most preferably is hydrogen or ammonium ion. In the absence of linker (R₅), it gives the general structure of cardiolipin (FIG. 1).

The invention provides a method for preparing cardiolipin or an analogue thereof of Formulas I, II, or III, comprising reacting an alcohol of the formula VIII with one or more phosphoramidite reagents and 2-O-protected glycerol or 2-O-subsituted glycerol VI in the presence of an acid catalyst. 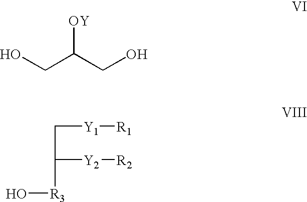
Y in formula VI is a hydroxyl protecting group, preferably alkyl group or the like, or a silyl protecting group. In Formula VIII, R₁, R₂, R₃, Y₁, and Y₂, can be as indicated above with respect to Formulas I, II, or III. In accordance with the inventive method, the acid catalyst can be any suitable catalyst that can facilitate the reaction. Examples of such catalysts include 4,5-dichloroimidazole, 1H-tetrazole, 5-(4-nitrophenyl)-1H-tetrazole, 5-(3,5-dinitrophenyl)-1H-tetrazole, N-methylimidazolium triflate, and N-methylimidazolium perchlorate, 4,5-dicyanoimidazole, 5-ethylthio-1H-tetrazole, and 5-methylthio-1H-tetrazole. Preferred catalysts are 4,5-dichloroimidazole or 1H-tetrazole. In accordance with the inventive method, the coupling phosphoramidites can have formula IV or V:

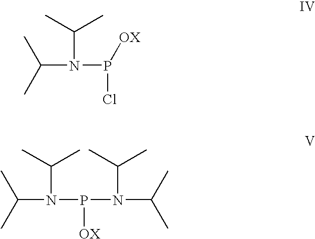

In another embodiment, the invention provides a method for preparing cardiolipin or an analogue thereof of formulas I, II, or III; comprising reacting 2-O protected glycerol with one or more phosphotriesters in the presence of pyridinium tribromide. Preferred phosphotriesters can be produced by reacting an alcohol of formula VIII with phosphoramidite of general formula VII.

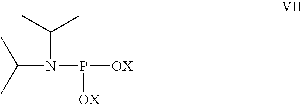

X in Formulas IV, V, or VII is a phosphate protecting group, preferably a benzyl group or 2-cyanoethyl or silyl group. Other examples of suitable protecting groups include alkyl phosphates including ethyl, cyclohexyl, t-butyl; 2-substituted ethyl phosphates including 2-cyanoethyl, 4-cyano-2-butenyl, 2-(methyldiphenylsilyl)ethyl, 2-(trimethylsilyl)ethyl, 2-(triphenylsilyl)ethyl; haloethyl phosphates including 2,2,2-trichloroethyl, 2,2,2-tribromoethyl, 2,2,2-trifluoroethyl; benzyl phosphates including 4-chlorobenzyl, fluorenyl-9-methyl, diphenylmethyl and amidates.

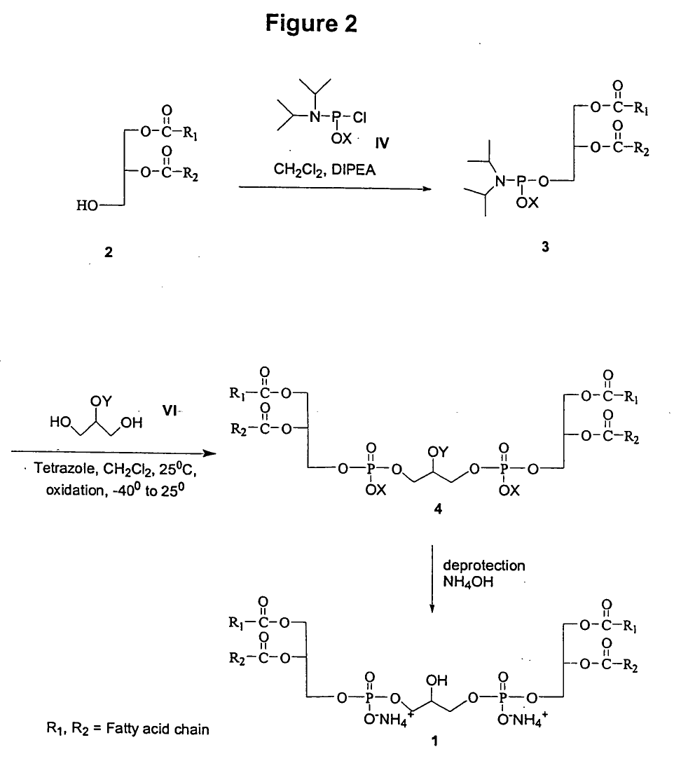

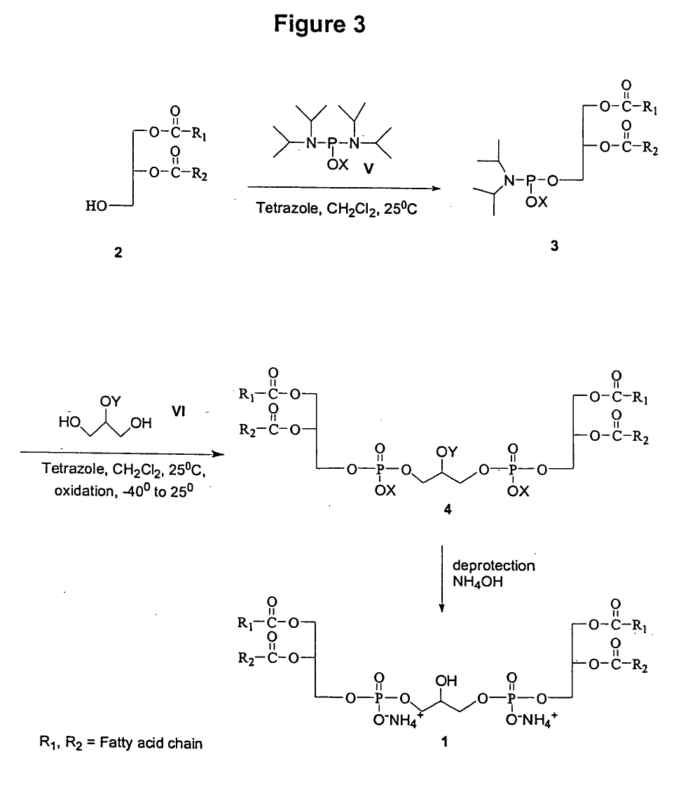

A general sequence of reactions for the synthesis of compound of invention is illustrated in FIGS. 2 & 3. The present invention provides a method for preparing cardiolipin I having varying fatty acid chain lengths comprising (a) reacting an optically pure 1,2-O-diacyl-sn-glycerol 2 with one or more phosphoramidite reagent(s) of the general formula IV (FIG. 2) or V (FIG. 3) (b) coupling the product of (a) 3 with a 2-O-protected glycerol VI in a chlorinated solvent (for example dichloromethane, chloroform or like) followed by oxidation with m-chloroperoxybenzoic acid (m-CPBA) results in the production of a protected cardiolipin 4. Thereafter, deprotecting the protected cardiolipin followed by conversion to ammonium salt will result in the production of cardiolipin 1 (ammonium salt).

For reaction with optically pure 1,2-O-diacyl-sn-glycerol 2, any suitable phosphoramidite reagent or methodology may be used, such as is described in, for example Browne et al., supra. Examples of suitable phosphoramidite reagents include (benzyloxy)(N,N-diisopropylamino)chlorophosphine (see, e.g., Prestwich et al. J. Am. Chem. Soc. 1991, 113, 1822-1825), benzyloxybis (diisopropylamino) phosphine (see, e.g., Dreef et al. Tetrahedron Lett. 1988, 29, 6513-6516), 2-cyanoethyl-N,N,N,N-tetraisopropylphosphoramidite (see, e.g., Browne et al. J. Chem. Soc. Perkin Trans. 1. 2000, 653-657.), (2-cyanoethyl)(N,N-diisopropylamino)chlorophosphine (see, e.g., Prestwich et al. J. Org. Chem. 1998, 63, 6511-6522), difluorenyl diisopropylphosphoramidite (see, e.g., Watanabe et al. Tetrahedron Lett. 1997, 38, 7407-7410), methyl-N,N,N,N tetraisopropylphosphorodiamidite (see, e.g., Murakami et al. J. Org. Chem. 1999, 64, 648-651), dimethyl N,N-diisopropylphosphoramidite (see, e.g., Watanabe et al. Tetrahedron Lett. 1993, 34, 497-500), dibenzyl diisopropylphosphoramidite (see, e.g., Watanabe et al. Tetrahedron Lett. 2000, 41, 8509-8512), di-tert-butyl-N,N-diisopropylphosphoramidite (see, e.g., Lindberg et al. J. Org. Chem. 2002, 67, 194-199.), 2-(diphenylmethylsilyl)ethyl-N,N,N,N-tetraisopropylphosphoramidite (see, e.g., Chevallier et al. Org. Lett. 2000, 2, 1859-1861), (N-trifluoroacetylamino) butyl and (N-trifluoroacetylamino) pentyl-N,N,N,N-tetraisopropylphosphoramidites (see, e.g., wilk et al. J. Org. Chem. 1997, 62, 6712-6713).

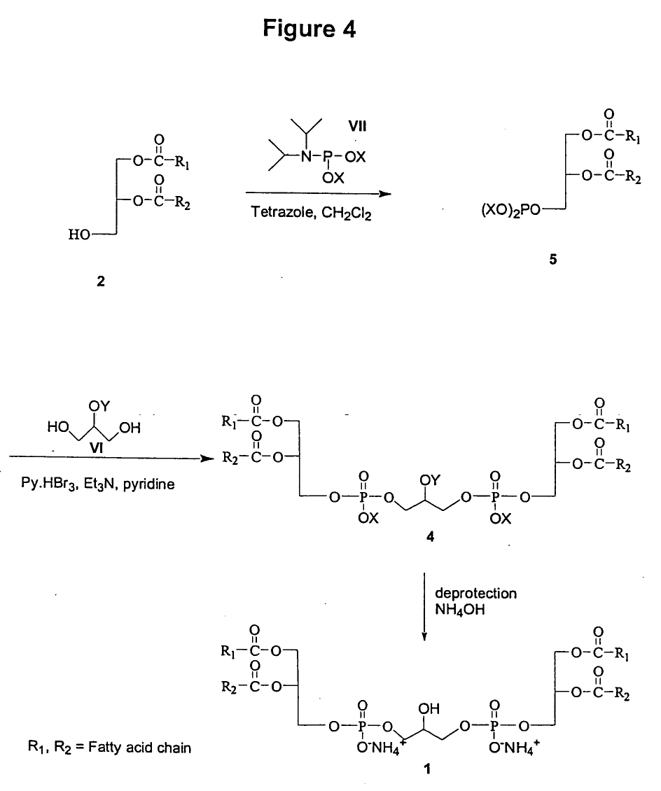

Another embodiment of the present invention is depicted in FIG. 4. In this method the optically pure 1,2-O-diacyl-sn-glycerol 2 can be phosphorylated using phosphoramidite VII to yield phosphite triesters 5 which can be coupled with any suitable 2-O-protected glycerol VI, such as, for example, benzyloxy 1,3-propanediol or 2-levulinoyl-1,3-propanediol using pyridinium perbromide and phosphonium salt methodology (see, e.g., Watanabe et al., supra) to get protected cardiolipin 4. The preferred coupling reagent in this context of synthetic methods is dibenzyl diisopropylphosphoramidite.

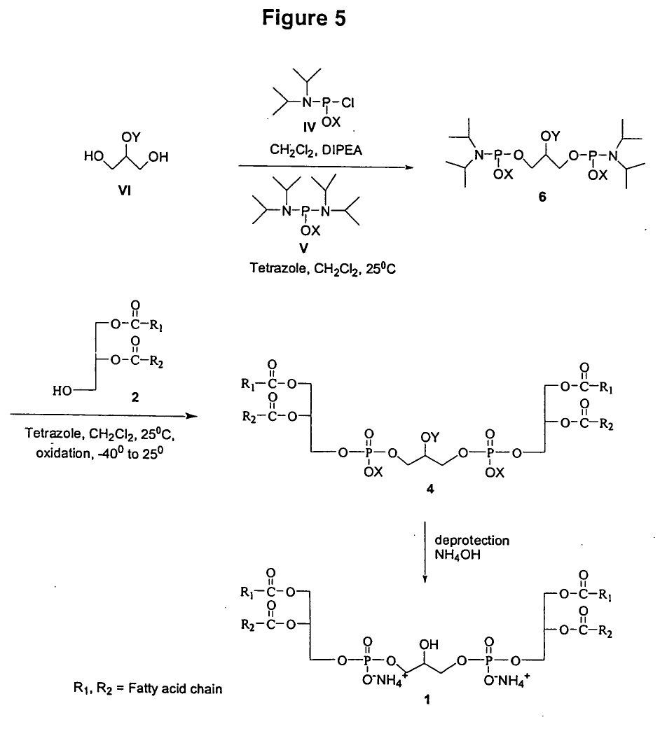

In an alternative strategy set forth in FIG. 5, the inventive method comprises (a) reacting a 2-O-protected glycerol VI with one or more phosphoramidite reagents IV or V, wherein a phosphorylating agent 6 is produced, (b) reacting the phosphorylating agent 6 with an optically pure 1,2-O-diacyl-sn-glycerol 2 followed by oxidation with m-CPBA, wherein a protected cardiolipin 4 is produced, and (c) deprotecting the protected cardiolipin, such that the cardiolipin is prepared. Suitable phosphoramidite reagents and 2-O-protected glycerols for use in this aspect of the inventive method are described above.

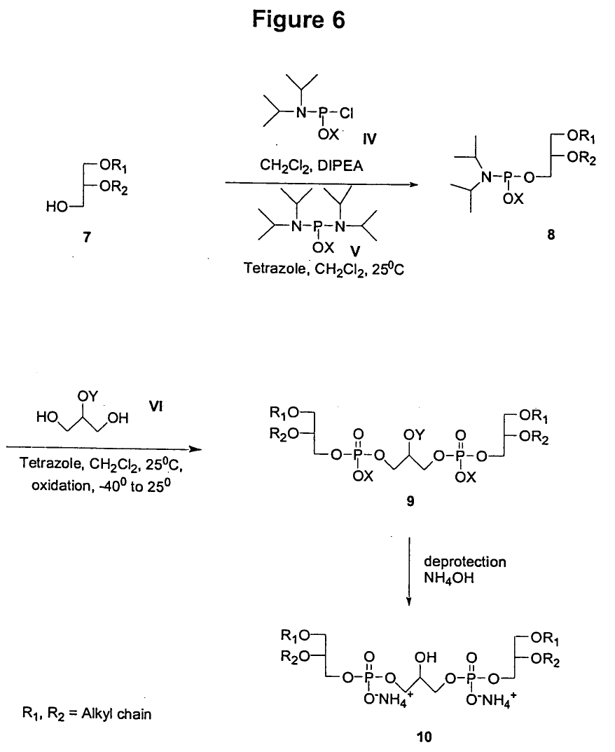

Another embodiment of the present invention, represented in FIG. 6 leads to ether analogs of cardiolipin, wherein the acyl groups are replaced by alkyl chain. Accordingly (a) 1,2-O-dialkyl-sn-glycerol 7 is treated with phosphoramidites IV or V wherein a phosphorylating agent 8 is produced, (b) reacting the phosphorylating agent with a 2-O-protected glycerol VI followed by oxidation, wherein a protected cardiolipin 9 is produced, and (c) deprotecting the protected cardiolipin, such that the ether analog of cardiolipin 10 is produced.

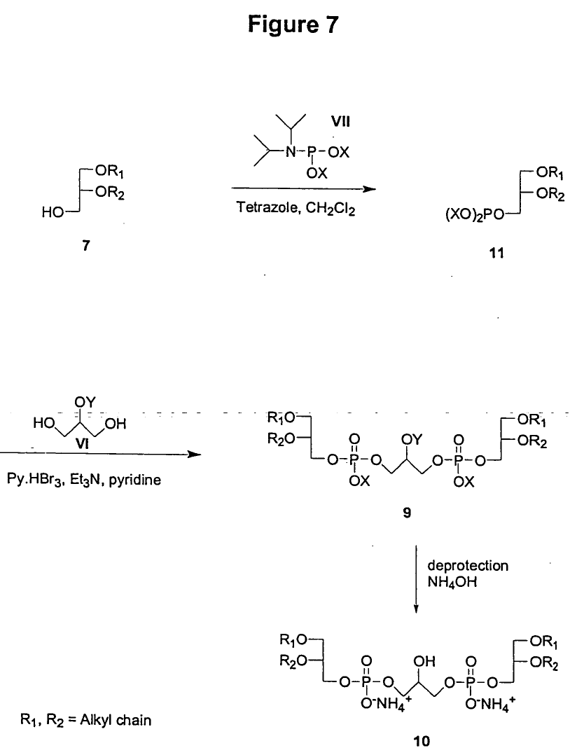

Another embodiment of the present invention is depicted in FIG. 7. In this method the optically pure 1,2-O-dialkyl-sn-glycerol 7 can be phosphorylated using phosphoramidite VII to yield phosphite triesters 11 which can be coupled with any suitable 2-O-protected glycerol VI, such as, for example, benzyloxy 1,3-propanediol or 2-levulinoyl-1,3-propanediol using pyridinium perbromide and phosphonium salt methodology (see, e.g., Watanabe et al., supra) to get protected cardiolipin ether analog 9. The preferred coupling reagent in this context of synthetic methods is dibenzyl diisopropylphosphoramidite.

The invention described above is an elegant and efficient method of synthesizing cardiolipin. The routes are short and proceed in good overall yield. The deprotection can be accomplished by a method depending on the protecting group. For example a benzyl group can be removed by catalytic hydrogenolysis or by treatment with NaI, 2-cyanoethyl and fluorenylmethyl groups by treatment with a tertiary base such as triethylamine, a silyl group can be deprotected with fluoride ion or acidic medium, a levulinoyl group by hydrazinolysis.

The synthetic methods described herein can be modified in any suitable manner. For example, phosphoramidites and phosphate esters can be prepared using a variety of acid catalysts, including 4,5-dichloroimidazole (see, e.g., Browne et al.), 5-(4-nitrophenyl)-1H-tetrazole, 5-(3,5-dinitrophenyl)-1H-tetrazole, N-methylimidazolium triflate, and N-methylimidazolium perchlorate (see, e.g., Moriguchi et al.). Likewise, tert-butylhydroperoxide can be used as an alternative oxidant. The described methods can be further modified in any suitable manner known in the art.

The inventive method can be used to prepare cardiolipin species comprising fatty acid/alkyl chains of varying length and saturation/unsaturation. The general structure of a phospholipid fatty acid comprises a hydrocarbon chain and a carboxylic acid group. In general, the length of the fatty acid hydrocarbon chain ranges from about 2 to about 34 carbon atoms and can be saturated or unsaturated. However, the carbon chain is more typically between about 12 and about 24 carbon atoms. In some embodiments, it is desirable for the hydrocarbon chain to comprise, for example, at least about 5 carbon atoms or at least about 10 carbon atoms or even at least about 15 carbon atoms. Typically, the length of the fatty acid hydrocarbon is less than about 24 carbon acids, such as less than about 24 carbon atoms, or even less than about 20 carbon atoms.

The invention also provides a cardiolipin or cardiolipin analogue prepared in accordance with the inventive method. Most preferably, the cardiolipin prepared by the inventive method comprises a short fatty acid chain (i.e., a “short chain cardiolipin”), and the invention provides a short chain cardiolipin. A short fatty acid chain comprises between about 2 and between about 14 carbon atoms, and can have between about 4 (or about 6) and about 12 carbon atoms, such as between about 8 and about 10 carbon atoms. Alternatively, the cardiolipin produced by the inventive method can comprise a long chain fatty acid chain (i.e., a “long chain cardiolipin”). A long fatty acid chain comprises between about 14 and about 34 carbon atoms, such as between about 14 (or between about 20) and about 24 carbon atoms. The inventive method is not limited to the production of short or long chain cardiolipin species exclusively. Indeed, a cardiolipin containing fatty acid/alkyl chains of intermediate length can also be prepared by the inventive method.

Phospholipid fatty acids typically are classified by the number of double and/or triple bonds in the hydrocarbon chain (i.e., unsaturation). A saturated fatty acid does not contain any double or triple bonds, and each carbon in the chain is bound to the maximum number of hydrogen atoms. The degree of unsaturation of a fatty acid depends on the number of double or triple bonds present in the hydrocarbon chain. In this respect, a monounsaturated fatty acid contains one double bond, whereas a polyunsaturated fatty acid contains two or more double bonds (see, e.g., Oxford Dictionary of Biochemistry and Molecular Biology, rev. ed., A. D. Smith (ed.), Oxford University Press (2000), and Molecular Biology of the Cell, 3^{rd} ed., B. A. Alberts (ed.), Garland Publishing, New York (1994)). The fatty acid chains of the cardiolipin are prepared by the inventive method, whether short or long, also can be saturated or unsaturated.

The described methods can be used to prepare a variety of novel cardiolipin molecules. For example, the methods can be used to prepare cardiolipin variants in pure form containing short or long fatty acid side chains. Preferred fatty acids range from carbon chain lengths of about C₂ to C₃₄, preferably between about C₄ and about C₂₄, and include tetranoic acid (C_{4:0}), pentanoic acid (C_{5:0}), hexanoic acid (C_{6:0}), heptanoic acid (C_{7:0}), octanoic acid (C_{8:0}), nonanoic acid (C_{9:0}), decanoic acid (C_{10:0}), undecanoic acid (C_{11:0}), dodecanoic acid (C_{12:0}), tridecanoic acid (C_{13:0}), tetradecanoic (myristic) acid (C_{14:0}), pentadecanoic acid (C_{15:0}), hexadecanoic (palmatic) acid (C_{16:0}), heptadecanoic acid (C_{17:0}), octadecanoic (stearic) acid (C_{18:0}), nonadecanoic acid (C_{19:0}), eicosanoic (arachidic) acid (C_{20:0}), heneicosanoic acid (C_{21:0}), docosanoic (behenic) acid (C_{22:0}), tricosanoic acid (C_{23:0}), tetracosanoic acid (C_{24:0}), 10-undecenoic acid (C_{11:1}), 11-dodecenoic acid (C_{12:1}), 12-tridecenoic acid (C_{13:1}), myristoleic acid (C_{14:1}), 10-pentadecenoic acid (C_{15:1}), palmitoleic acid (C_{16:1}), oleic acid (C_{18:1}), linoleic acid (C_{18:2}), linolenic acid (C_{18:3}), eicosenoic acid (C_{20:1}), eicosdienoic acid (C_{20:2}), eicosatrienoic acid (C_{20:3}), arachidonic acid (cis-5,8,11,14-eicosatetraenoic acid), and cis-5,8,11,14,17-eicosapentaenoic acid, among others. For ether analogs, the alkyl chain will also range from C₂ to C₃₄ preferably between about C₄ and about C₂₄. Other fatty acid chains also can be employed as R₁ and/or R₂ substituents. Examples of such include saturated fatty acids such as ethanoic (or acetic) acid, propanoic (or propionic) acid, butanoic (or butyric) acid, hexacosanoic (or cerotic) acid, octacosanoic (or montanic) acid, triacontanoic (or melissic) acid, dotriacontanoic (or lacceroic) acid, tetratriacontanoic (or gheddic) acid, pentatriacontanoic (or ceroplastic) acid, and the like; monoethenoic unsaturated fatty acids such as trans-2-butenoic (or crotonic) acid, cis-2-butenoic (or isocrotonoic) acid, 2-hexenoic (or isohydrosorbic) acid, 4-decanoic (or obtusilic) acid, 9-decanoic (or caproleic) acid, 4-dodecenoic (or linderic) acid, 5-dodecenoic (or denticetic) acid, 9-dodecenoic (or lauroleic) acid, 4-tetradecenoic (or tsuzuic) acid, 5-tetradecenoic (or physeteric) acid, 6-octadecenoic (or petroselenic) acid, trans-9-octadecenoic (or elaidic) acid, trans-11-octadecenoic (or vaccinic) acid, 9-eicosenoic (or gadoleic) acid, 11-eicosenoic (or gondoic) acid, 11-docosenoic (or cetoleic) acid, 13-decosenoic (or erucic) acid, 15-tetracosenoic (or nervonic) acid, 17-hexacosenoic (or ximenic) acid, 21-triacontenoic (or lumequeic) acid, and the like; dienoic unsaturated fatty acids such as 2,4-pentadienoic (or β-vinylacrylic) acid, 2,4-hexadienoic (or sorbic) acid, 2,4-decadienoic (or stillingic) acid, 2,4-dodecadienoic acid, 9,12-hexadecadienoic acid, cis-9, cis-12-octadecadienoic (or α-linoleic) acid, trans-9, trans-12-octadecadienoic (or linlolelaidic) acid, trans-10,trans-12-octadecadienoic acid, 11,14-eicosadienoic acid, 13,16-docosadienoic acid, 17,20-hexacosadienoic acid and the like; trienoic unsaturated fatty acids such as 6,10,14-hexadecatrienoic (or hiragonic) acid, 7,10,13-hexadecatrienoic acid, cis-6, cis-9-cis-12-octadecatrienoic (or γ-linoleic) acid, trans-8, trans-10-trans-12-octadecatrienoic (or β-calendic) acid, cis-8, trans-10-cis-12-octadecatrienoic acid, cis-9, cis-12-cis-15-octadecatrienoic (or α-linolenic) acid, trans-9, trans-12-trans-15-octadecatrienoic (or α-linolenelaidic) acid, cis-9, trans-11-trans-13-octadecatrienoic (or aleostearic) acid, trans-9, trans-11-trans-13-octadecatrienoic (or β-eleostearic) acid, cis-9, trans-11-cis-13-octadecatrienoic (or punicic) acid, 5,8,11-eicosatrienoic acid, 8,11,14-eicosatrienoic acid and the like; tetraenoic unsaturated fatty acids such as 4,8,11,14-hexadecatetraenoic acid, 6,9,12,15-hexadecatetraenoic acid, 4,8,12,15-octadecatetraenoic (or moroctic) acid, 6,9,12,15-octadecatetraenoic acid, 9,11,13,15-octadecatetraenoic (or α- or β-parinaric) acid, 9,12,15,18-octadecatetraenoic acid, 4,8,12,16-eicosatetraenoic acid, 6,10,14,18-eicosatetraenoic acid, 4,7,10,13docasatetraenoic acid, 7,10,13,16-docosatetraenoic acid, 8,12,16,19-docosatetraenoic acid and the like; penta- and hexa-enoic unsaturated fatty acids such as 4,8,12,15,18-eicosapentaenoic (or timnodonic) acid, 4,7,10,13,16-docosapentaenoic acid, 4,8,12,15,19-docosapentaenoic (or clupanodonic) acid, 7,10,13,16,19-docosapentaenoic, 4,7,10,13,16,19-docosahexaenoic acid, 4,8,12,15,18,21-tetracosahexaenoic (or nisinic) acid and the like; branched-chain fatty acids such as 3-methylbutanoic (or isovaleric) acid, 8-methyldodecanoic acid, 10-methylundecanoic (or isolauric) acid, 11-methyldodecanoic (or isoundecylic) acid, 12-methyltridecanoic (or isomyristic) acid, 13-methyltetradecanoic (or isopentadecylic) acid, 14-methylpentadecanoic (or isopalmitic) acid, 15-methylhexadecanoic, 10-methylheptadecanoic acid, 16-methylheptadecanoic (or isostearic) acid, 18-methylnonadecanoic (or isoarachidic) acid, 20-methylheneicosanoic (or isobehenic) acid, 22-methyltricosanoic (or isolignoceric) acid, 24-methylpentacosanoic (or isocerotic) acid, 26-methylheptacosanoic (or isomonatonic) acid, 2,4,6-trimethyloctacosanoic (or mycoceranic or mycoserosic) acid, 2-methyl-cis-2-butenoic(angelic)acid, 2-methyl-trans-2-butenoic (or tiglic) acid, 4-methyl-3-pentenoic (or pyroterebic) acid and the like.

The term ‘hydroxyl protecting group’ used herein the invention refers to the commonly used protecting groups disclosed by T. W. Greene and P. G. Wuts, Protective Groups in Organic Synthesis, 3 rd edition, John Wiley & Sons, New York (1999). Such protecting groups include methyl ether, substituted methyl ethers including methoxymethyl, benzyloxymethyl, p-methoxybenzyloxymethyl, 2-methoxyethoxymethyl, tetrahydropyranyl, tetrahydrofuranyl ethers; substituted ethyl ethers like 1-ethoxyethyl, 1-methyl-1-benzyloxyethyl, allyl, propargyl; benzyl and substituted benzyl ethers including p-methoxybenzyl, 3,4-dimethoxybenzyl, triphenylmethyl; silyl ethers including trimethylsilyl, triethylsilyl, t-butyldimethylsilyl, t-butyldiphenylsilyl, diphenylmethylsilyl; esters including formate, acetate, chloroacetate, dichloroacetate, trichloroacetate, benzoate, levulinylate and carbonates.

The term ‘phosphate protecting group’ used herein the invention refers to the commonly used protecting groups described by T. W. Greene and P. G. Wuts, Protective Groups in Organic Synthesis, 3 rd edition, John Wiley & Sons, New York (1999). Such protecting groups include alkyl phosphates including methyl, ethyl, cyclohexyl, t-butyl; 2-substituted ethyl phosphates including 2-cyanoethyl, 4-cyano-2-butenyl, 2-(methyldiphenylsilyl)ethyl, 2-(trimethylsilyl)ethyl, 2-(triphenylsilyl)ethyl; haloethyl phosphates including 2,2,2-trichloroethyl, 2,2,2-tribromoethyl, 2,2,2-trifluoroethyl; benzyl phosphates including 4-chlorobenzyl, fluorenyl-9-methyl, diphenylmethyl and amidates.

The cardiolipin molecules described herein and cardiolipins produced by the inventive method can be used in lipid formulations, such as liposomal compositions. Complexes, emulsions and other formulations including the inventive cardiolipin also are within the scope of the present invention. Such formulations according to the present invention can be prepared by any suitable technique. The invention provides a method for preparing a liposome or other lipid composition, comprising preparing a cardiolipin or cardiolipin analogue as described herein and including the cardiolipin or cardiolipin analogue in a lipid formulation, such as a liposome. The invention also includes such lipid compositions including the inventive cardiolipin and/or cardiolipin analogues.

In addition to the inventive cardiolipin, the liposomal composition, complex, emulsion and the like can include other lipids. Thus, for example, the composition can include one or more phosphatidylcholines, such as, for example, dimyristoylphosphatidylcholine, distearoylphosphatidylcholine, dioleylphosphatidylcholine, dipalmitoylphosphatidylcholine, diarachidonoylphosphatidylcholine, egg phosphatidylcholine, soy phosphatidylcholine, hydrogenated soy phosphatidylcholine, and mixtures thereof. Alternatively or additionally, the composition can include one or more phosphatidylglycerols, such as dimyristoylphosphatidylglycerol, distearoylphosphatidylglycerol, dioleylphosphatidylglycerol, dipalmitoylphosphatidylglycerol, diarachidonoylphosphatidylglycerol, and mixtures thereof. Alternatively or additionally, the composition can include one or more sterols, such as cholesterol, derivatives of cholesterol, coprostanol, cholestanol, cholestane, cholesterol hemisuccinate, cholesterol sulfate, and mixtures thereof. Preferably, in addition to the cardiolipin or cardiolipin analogue, the composition includes a phosphatidylcholine, a sterol, and a tocopherol (e.g., α-tocopherol).

In addition to the cardiolipin and, optionally, other lipids, the composition also can include stabilizers, absorption enhancers, antioxidants, phospholipids, biodegradable polymers and medicinally active agents among other ingredients. In some embodiments, it is preferable for the inventive composition, especially liposomal composition, to include one or more targeting agents, such as carbohydrate or a protein or other ligand that binds to a specific substrate, for example, that recognize cellular receptors. The inclusion of such agents (such as a carbohydrate or one or more proteins selected from groups of proteins consisting of antibodies, antibody fragments, peptides, peptide hormones, receptor ligands such as an antibody to a cellular receptor and mixtures thereof) can facilitate targeting a liposome to a predetermined tissue or cell type.

For medicinal use, the composition also can include one or more active agents. A single active agent can be included, or a mixture of active agents (e.g., two or more active agents) can be included within the composition. Active agents (or “drugs”) can be present in any suitable manner in the composition. For example, they can be complexed with the cardiolipin or cardiolipin analogue in the composition. Additionally, or alternatively, one or more active agents can be entrapped within liposomes, when the composition is a liposomal composition.

Active agents which are compatible with the present invention include, for example, agents which act on the peripheral nerves, adrenergic receptors, cholinergic receptors, the skeletal muscles, the cardiovascular system, smooth muscles, the blood circulatory system, synaptic sites, neuroeffector junctional sites, endocrine and hormone systems, the immunological system, the reproductive system, the skeletal system, the alimentary and excretory systems, the histamine system and the central nervous system. Suitable agents may be selected from, for example, proteins, enzymes, hormones, nucleotides (including sense and antisense oligonucleotides (see, e.g., U.S. Pat. No. 6,126,965), polynucleotides, nucleoproteins, polysaccharides, glycoproteins, lipoproteins, polypeptides, steroids. Active agents can be analgesics, anesthetics, anti-arrythmic agents, antibiotics, antiallergic agents, antifungal agents, anticancer agents, anticoagulants, antidepressants, antidiabetic agents, anti-epilepsy agents, anti-inflammatory corticosteroids, agents for treating Alzheimers or Parkinson's disease, antiulcer agents, anti-protozoal agents, anxiolytics, thyroids, anti-thyroids, antivirals, anoretics, bisphosphonates, cardiac inotropic agents, cardiovascular agents, corticosteroids, diuretics, dopaminergic agents, gastrointestinal agents, hemostatics, hypercholesterol agents, antihypertensive agents (e.g., dihydropyridines), antidepressants, and cox-2 inhibitors, immunosuppressive agents, anti-gout agents, anti-malarials, steroids, terpinoids, triterpines, retinouds; anti-ulcer H2-receptor antagonists, hypoglycemic agents, moisturizers, cosmetics, anti-migraine agents, antimuscarinic agents, antiinflammatory agents, such as agents for treating rheumatology, arthritis, psoriasis, inflammatory bowel disease, Crohn's disease; or agents for treating demyelinating diseases including multiple sclerosis, ophthalmic agents, vaccines (e.g., against pneumonia, hepatitis A, hepatitis B, hepatitis C, cholera toxin B subunit, influenza virus, typhoid, plasmodium falciparun, diptheria, tetanus, HSV, tuberculosis, HIV, SARS virus, pordetela pertussis, measles, mumps and rubella vaccine (MMV), bacterial toxoids, vaccinea virus, adenovirus, canary, polio virus, bacillus calmette guerin (BCG), klebsiella pneumonia, etc.), histamine receptor antagonists, hypnotics, kidney protective agents, lipid regulating agents, muscle relaxants, neuroleptics, neurotropic agents, opioid agonists and antagonists, parasympathomimetics, protease inhibitors, prostglandins, sedatives, sex hormones (e.g., estrogen, androgen), stimulants, sympathomimetics, vasodilators and xanthins and synthetic analogs of these species. The therapeutic agents can be nephrotoxic, such as cyclosporins and amphotericin B, or cardiotoxic, such as amphotericin B and paclitaxel. Exemplary anticancer agents include melphalan, chlormethine, extramustinephosphate, uramustine, ifosfamide, mannomustine, trifosfamide, streptozotocin, mitobronitol, mitoxantrone (see., e.g., published international patent application WO 02/32400), methotrexate, fluorouracil, cytarabine, tegafur, idoxide, taxanes (e.g., taxol, paclitaxel, etc., see published international patent application WO 00/01366), daunomycin, daunorubicin, bleomycin, amphotericin, carboplatin, cisplatin, paclitaxel, BCNU, vinca alkaloids (e.g., vincristine, vinorelbine (see, e.g., published international patent application WO 03/018018), and the like) camptothecin and derivatives thereof (e.g., SN38 (see, e.g., published international patent application WO 02/058622), irinotecan (see, e.g., published international patent application WO 03/030864), and the like), anthracyclines, antibodies, cytoxines, doxorubicin, etopside, cytokines, ribozymes, interferons, oligonucleotides and functional derivatives of the foregoing. Additional examples of drugs which may be delivered according to the method include, prochlorperzine edisylate, ferrous sulfate, aminocaproic acid, mecamylamine hydrochloride, procainamide hydrochloride, amphetamine sulfate, methamphetamine hydrochloride, benzamphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, thiethylperzine maleate, anisindone, diphenadione erythrityl tetranitrate, digoxin, isoflurophate, acetazolamide, methazolamide, bendroflumethiazide, chloropromaide, tolazamide, chlormadinone acetate, phenaglycodol, allopurinol, aluminum aspirin, methotrexate, acetyl sulfisoxazole, erythromycin, hydrocortisone, hydrocorticosterone acetate, cortisone acetate, dexamethasone and its derivatives such as betamethasone, triamcinolone, methyltestosterone, 17-S-estradiol, ethinyl estradiol, ethinyl estradiol 3-methyl ether, prednisolone, 17α-hydroxyprogesterone acetate, 19-norprogesterone, norgestrel, norethindrone, norethisterone, norethiederone, progesterone, norgesterone, norethynodrel, aspirin, indomethacin, naproxen, fenoprofen, sulindac, indoprofen, nitroglycerin, isosorbide dinitrate, propranolol, timolol, atenolol, alprenolol, cimetidine, clonidine, imipramine, levodopa, chlorpromazine, methyldopa, dihydroxyphenylalanine, theophylline, calcium gluconate, ketoprofen, ibuprofen, cephalexin, erythromycin, haloperidol, zomepirac, ferrous lactate, vincamine, diazepam, phenoxybenzamine, diltiazem, milrinone, mandol, quanbenz, hydrochlorothiazide, ranitidine, flurbiprofen, fenufen, fluprofen, tolmetin, aldlofenac, mefenamic, flufenamic, difuinal, nimodipine, nitrendipine, nisoldipine, nicardipine, felodipine, lidoflazine, tiapamil, gallopamil, amlodipine, mioflazine, lisinolpril, enalapril, enalaprilat captopril, ramipril, famotidine, nizatidine, sucralfate, etintidine, tetratolol, minoxidil, chlordiazepoxide, diazepam, amitriptyline, and imipramine. Further examples are proteins and peptides which include, but are not limited to, bone morphogenic proteins, insulin, colchicine, glucagon, thyroid stimulating hormone, parathyroid and pituitary hormones, digestive hormones, calcitonin, renin, prolactin, corticotrophin, thyrotropic hormone, follicle stimulating hormone, chorionic gonadotropin, gonadotropin releasing hormone, bovine somatotropin, porcine somatotropin, oxytocin, vasopressin, GRF, somatostatin, lypressin, pancreozymin, luteinizing hormone, LHRH, LHRH agonists and antagonists, leuprolide, interferons (e.g., consensus interferon, interferon α-2a, interferon α-2b, α-, β-, or γ-interferons), interleukins, growth hormones such as human growth hormone and its derivatives such as methione-human growth hormone and des-phenylalanine human growth hormone, bovine growth hormone and porcine growth hormone, fertility inhibitors such as the prostaglandins, fertility promoters, growth factors such as insulin-like growth factor, coagulation factors, pancreas hormone releasing factor, analogs and derivatives of these compounds, and pharmaceutically acceptable salts of these compounds, or their analogs or derivatives. The therapeutic agent can be a mixture of drugs or agents (e.g., two or more agents) that can be beneficially co-administered in the liposome formulation.

Generally, liposomes can have a net neutral, negative or positive charge. For example, positive liposomes can be formed from a solution containing phosphatidylcholine, cholesterol, cardiolipin and enough stearylamine to overcome the net negative charge of cardiolipin. Negative liposomes can be formed from solutions containing phosphatidyl choline, cholesterol, and/or cardiolipin variants prepared by the methods described herein.

The liposomes of the present invention can be multi or unilamellar vesicles depending on the particular composition and procedure to make them. Liposomes can be prepared to have substantially homogeneous sizes in a selected size range, such as about 1 micron or less, or about 500 nm or less, about 200 nm or less, or about 100 nm or less. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane.

The liposomal (or other lipid) composition can be in any desired form. For example, for pharmaceutical use, the composition can be ready for administration to a patient. Alternatively, the composition can be in dried or lyophilized form. Where the composition is dried or lyophilized, preferably the composition includes a cryoprotectant as well. Suitable cryoprotectants include, for example, sugars such as trehalose, maltose, lactose, sucrose, glucose, and dextran, with the most preferred sugars from a performance point of view being trehalose and sucrose. Other more complicated sugars can also be used, such as, for example, aminoglycosides, including streptomycin and dihydrostreptomycin.

Any suitable method can be employed to form the liposomes. For example, lipophilic liposome-forming ingredients, such as phosphatidylcholine, a cardiolipin prepared by the methods described above, cholesterol and α-tocopherol can be dissolved or dispersed in a suitable solvent or combination of solvents and dried. Suitable solvents include any non-polar or slightly polar solvent, such as t-butanol, ethanol, methanol, chloroform, or acetone that can be evaporated without leaving a pharmaceutically unacceptable residue. Drying can be by any suitable means such as by lyophilization. The dehydration is typically achieved under vacuum and can take place either with or without prior freezing of the liposome preparation. Hydrophilic ingredients can be dissolved in polar solvents, including water.

Mixing the dried lipophilic ingredients with the hydrophilic mixture can form liposomes. Mixing the polar solution with the dry lipid film can be by any means that strongly homogenizes the mixture. Vortexing, magnetic stirring and/or sonicating can effect the homogenization.

Where active agents (or a mixture of active agents) are included in the liposomes, the invention provides a method for retaining a drug in a liposome. In accordance with the method, cardiolipin or cardiolipin analogue is prepared as described herein, and the cardiolipin or cardiolipin analogue and a drug or drugs (e.g., an active agent a mixture of active agents) is included within a liposome. For example, active agent(s) can be dissolved or dispersed in a suitable solvent and added to the liposome mixture prior to mixing. Typically hydrophilic active agents will be added directly to the polar solvent and hydrophobic active agents will be added to the nonpolar solvent used to dissolve the other ingredients but this is not required. The active agent could be dissolved in a third solvent or solvent mix and added to the mixture of polar solvent with the lipid film prior to homogenizing the mixture.

Liposomes can be coated with a biodegradable polymers such as sucrose, epichlorohydrin, branched hydrophilic polymers of sucrose, polyethylene glycols, polyvinyl alcohols, methoxypolyethylene glycol, ethoxypolyethylene glycol, polyethylene oxide, polyoxyethylene, polyoxypropylene, cellulose acetate, sodium alginate, N,N-diethylaminoacetate, block copolymers of polyoxyethylene and polyoxypropylene, polyvinyl pyrrolidone, polyoxyethylene X-lauryl ether wherein X is from 9 to 20, and polyoxyethylene sorbitan esters.

Antioxidants can be included in the liposomal composition or other lipid composition. Suitable antioxidants include compounds such as ascorbic acid, tocopherol, and deteroxime mesylate.

Absorption enhancers can be included in the liposomal composition or other lipid composition. Suitable absorption enhancers include Na-salicylate-chenodeoxy cholate, Na deoxycholate, polyoxyethylene 9-lauryl ether, chenodeoxy cholate-deoxycholate and polyoxyethylene 9-lauryl ether, monoolein, Na tauro-24,25dihydrofusidate, Na taurodeoxycholate, Na glycochenodeoxycholate, oleic acid, linoleic acid, linolenic acid. Polymeric absorption enhancers can also be included such as polyoxyethylene ethers, polyoxyethylene sorbitan esters, polyoxyethylene 10-lauryl ether, polyoxyethylene 16-lauryl ether, azone (1-dodecylazacycloheptane-2-one).

The inventive lipid (e.g., liposomal) composition also can include one or more pharmaceutically acceptably excipients. For example, pharmaceutically suitable excipients include solid, semi-solid or liquid diluents, fillers and formulation auxiliaries of all kinds. The invention also includes pharmaceutical preparations in dosage units. This means that the preparations are in the form of individual parts, for example vials, syringes, capsules, pills, suppositories, or ampoules, of which the content of the liposome formulation of active agent corresponds to a fraction or a multiple of an individual dose. The dosage units can contain, for example, 1, 2, 3, or 4 individual doses, or ½, ⅓, or ¼ of an individual dose. An individual dose preferably contains the amount of active agent which is given in one administration and which usually corresponds to a whole, a half, a third, or a quarter of a daily dose.

Tablets, dragees, capsules, pills, granules, suppositories, solutions, suspensions and emulsions, pastes, ointments, gels, creams, lotions, powders and sprays can be suitable pharmaceutical preparations. Suppositories can contain, in addition to the liposomal active agent, suitable water-soluble or water-insoluble excipients. Suitable excipients are those in which the inventive liposomal active agent is sufficiently stable to allow for therapeutic use, for example polyethylene glycols, certain fats, and esters or mixtures of these substances. Ointments, pastes, cream, and gels can also contain suitable excipients in which the liposomal active agent is stable. The composition also can be formulated for injection (e.g., intravenously, interstitially, intratumorally, etc) by the inclusion of one or more excipients (e.g., buffered saline) suitable for injection.

The active agent or its pharmaceutical preparations can be administered intravenously, subcutaneously, locally, orally, parenterally, intraperitoneally, and/or rectally or by direct injection into tumors or sites in need of treatment by such methods as are known or developed. Cardiolipin and cardiolipin-analog based formulations also can be administered topically, e.g., as a cream, skin ointment, dry skin softener, moisturizer, etc.

The invention provides for the use of the composition to prepare a medicament for the treatment of a disease. In this sense, the invention also provides a method for treating a human or animal disease. In accordance with the inventive method, the inventive composition is exposed to (administered to) a human or animal patient in need of such treatment. Where the composition also includes one or more active agents, the inventive method facilitates delivery of the active agent(s) to the patient.

The method can be used to administer one or more active agents. It is thought to be general for active agents that are stable in the presence of surfactants. Hydrophilic active agents are suitable and can be included in the interior of the liposomes such that the liposome bilayer creates a diffusion barrier preventing it from randomly diffusing throughout the body. Hydrophobic active agents are thought to be particularly well suited for use in the present method because they not only benefit by exhibiting reduced toxicity but they tend to be well solubilized in the lipid bilayer of liposomes.

Suitable diseases for treatment will depend on the selection of active agents, such as described herein. However a preferred disease is cancer, in which instance, at least one active agent incorporated into the composition is an anticancer agent. Chemotherapeutic agents are well suited for such use. Liposome formulations containing chemotherapeutic agents may be injected directly into the tumor tissue for delivery of the chemotherapeutic agent directly to cancer cells. In some cases, particularly after resection of a tumor, the liposome formulation can be implanted directly into the resulting cavity or may be applied to the remaining tissue as a coating. In cases in which the liposome formulation is administered after surgery, it is possible to utilize liposomes having larger diameters of about 1 micron since they do not have to pass through the vasculature.

In some embodiments, the method can be employed to treat diseases, disorders, or symptoms within patients even where the composition does not contain an active pharmaceutical agent other than cardiolipin. The invention provides for the use of cardiolipin to prepare a medicament to combat or treat such diseases, disorders, or symptoms. The invention further provides a method of treating such diseases, disorders, or symptoms within patients, and the effects of such diseases, disorders, or symptoms by administering to the patient a therapeutically effective amount of cardiolipin. Without being bound by any particular theory, it is believed that cardiolipin provides a beneficial antioxidant effect, which can alleviate the effects of many diseases, disorders, or symptoms. Examples of conditions that can be treated in accordance with the method include, for example, age-related diseases, atherosclerosis, diabetes, heart disease, ischemia, and skin disorders (e.g., acne, psoriasis, eczema, etc.). The method also can be employed to combat the effects of aging. For such use, the cardiolipin can be formulated as a liposomal or non-liposomal formulation (e.g., an emulsion, cream, etc.) as discussed herein and can include, in addition to cardiolipin, one or more pharmaceutically acceptable carriers. In use, the composition can be administered by any suitable route. For example, the composition can be administered dermally, intravenously, or by other desired route of administration.

The invention also is directed to methods of delivering active agents (or mixtures of active agents) to cells. The methods can be carried out by preparing liposomes that include active agents and cardiolipin variants/analogs as synthesized by the above disclosed methods. The liposomes are then delivered to a cell or cells, which can be in vitro or in vivo, as desired. In vivo administration can be achieved as described herein or as otherwise known to those of ordinary skill. For in vitro use, delivery of the active agent(s) can be carried out by adding the composition (e.g., liposomes) to the cell culture medium, for example.

The following examples further illustrate the invention but, of course, should not be construed as in any way limiting its scope.

### EXAMPLE 1

### Synthesis of Tetramyristoyl Cardiolipin

### 1A. 2-Benzyl-1,3-bis [(1,2-dimyristoyl-sn-glycero-3)-phosphoryl]glycerol dibenzyl ester

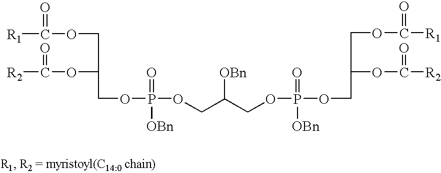

A solution of 1,2-dimyristoyl-sn-glycerol (10 g, 19.53 mmol), benzyl N,N-tetraisopropyl phosphoramidite (9.87 g, 29.29 mmol) and 1H-tetrazole (65 mL of 0.45 M sol in acetonitrile, 29.29 mmol) in CH₂Cl₂ (125 mL) was stirred at room temperature under argon for 3 h. A solution of 2-benzyloxy 1,3-propanediol (1.18 g, 6.47 mmol) in CH₂Cl₂ (20 mL) was added followed by 1H-tetrazole (37.7 mL of 0.45 M sol in acetonitrile, 16.85 mmol) and stirred for 3 h. The reaction mixture was cooled to −40° C. and tert-Butyl hydroperoxide (TBHP, 6.4 mL of 5-6 M sol in decane, 32.35 mmol) was added. After stirring at −40° C. for 30 minutes, the reaction mixture was warmed to room temperature, diluted with CH₂Cl₂ (250 mL), washed {saturated aq Na₂SO₃ (2×50 mL), saturated aq NaHCO₃ (2×50 mL), brine (2×50 mL)} dried (Na₂SO₄) and concentrated. The residue was purified on SiO₂ column (2:3 EtOAc:hexane) to give 6.68 g (69%) of protected cardiolipin as colorless syrup. TLC (SiO₂) hexane/EtOAc (3:2) R_{f}˜0.48. ¹H NMR δ (CDCl₃, 500 MHz) 0.88 (t, J=7.0 Hz, 12H), 1.22-1.34 (m, 80H), 1.52-1.66 (m, 8H), 2.22-2.29 (m, 8H), 3.70-3.78 (m, 1H), 4.01-4.16 (m, 10H), 4.22-4.28 (m, 2H), 4.60 (d, J=7.8 Hz, 2H), 5.01-5.05 (m, 4H), 5.15-5.18 (m, 2H), 7.28-7.36 (m, 15H).

### 1B. 1,3-bis [(1,2-dimyristoyl-sn-glycero-3)-phosphoryl]glycerol diammonium salt (Tetramyristoyl cardiolipin)

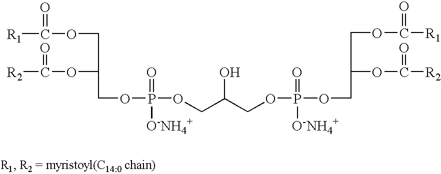

A solution of protected cardiolipin from 1A (2.5 g, 1.65 mmol) in tetrahydrofuran (40 mL) was hydrogenated at 50 psi over 10% Pd/C (900 mg) for 10 h. The catalyst was filtered off over celite bed, treated with 4 mL of 30% ammonia solution and concentrated, the residue was dissolved in CHCl₃, filtered through a 0.25μ filter and precipitated with acetone to give tetramyristoyl (C_{14:0}) cardiolipin (1.75 g, 83%) as a white solid. TLC (SiO₂) CHCl₃/MeOH/NH₄OH (6.5:2.5:0.5) R_{f}˜0.50. ¹H NMR δ (CDCl₃, 300 MHz) 0.88 (t, J=7.0 Hz, 12H), 1.22-1.34 (br s, 80H), 1.52-1.66 (m, 8H), 2.26-2.34 (m, 8H), 3.06 (bs, 1H), 3.82-3.98 (m, 9H), 4.12-4.18 (m, 2H), 4.35-4.42 (m, 2H), 5.14-5.24 (m, 2H), 7.41 (bs, 8H). ESI-MS (negative), m/z 1240.2 (M−2NH₄⁺+H⁺), 1011.9 (M−2NH₄⁺—RCOO⁻), 619.9 (M−2NH₄⁺)²⁻.

### EXAMPLE 2

### Synthesis of Tetralauroyl Cardiolipin

### 2A. 2-Benzyl-1,3-bis [(1,2-dilauroyl-sn-glycero-3)-phosphoryl]glycerol dibenzyl ester

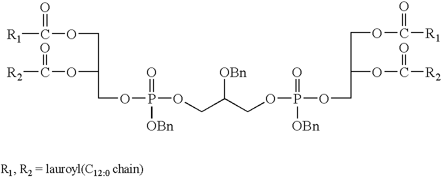

Method 1: A solution of 1,2-dilauroyl-sn-glycerol (2.2 g, 4.82 mmol), benzyl N, N-tetraisopropyl phosphoramidite (1.95 g, 5.78 mmol) and 1H-tetrazole (12.84 mL of 0.45 M sol in acetonitrile, 5.78 mmol) in CH₂Cl₂ (25 mL) was stirred at room temperature under argon for 3 h. A solution of 2-benzyloxy 1,3-propanediol (352 mg, 1.92 mmol) in CH₂Cl₂ (10 mL) was added followed by 1H-tetrazole (12.84 mL of 0.45 M sol in acetonitrile, 5.78 mmol) and stirred for 3 h. The reaction mixture was cooled to −40° C. and 3-Chloroperoxyperbenzoic acid (m-CPBA, 2.77 g, 9.64 mmol) was added in portions. After stirring at −40° C. for 30 minutes, the reaction mixture was warmed to room temperature, diluted with CH₂Cl₂ (150 mL), washed {saturated aq Na₂SO₃ (2×50 mL), saturated aq NaHCO₃ (2×50 mL), brine (2×50 mL)} dried (Na₂SO₄) and concentrated. The residue was purified on SiO₂ column (2:3 EtOAc:hexane) to give 1.68 g (62%) of protected cardiolipin as colorless syrup. TLC (SiO₂) hexane/EtOAc (3:2) R_{f}˜0.44. ¹H NMR δ (CDCl₃, 500 MHz) 0.88 (t, J=7.0 Hz, 12H), 1.22-1.34 (m, 64H), 1.52-1.66 (m, 8H), 2.22-2.29 (m, 8H), 3.70-3.78 (m, 1H), 4.01-4.16 (m, 10H), 4.22-4.28 (m, 2H), 4.60 (d, J=7.8 Hz, 2H), 5.01-5.05 (m, 4H), 5.15-5.18 (m, 2H), 7.28-7.36 (m, 15H).

Method 2: To a stirred solution of 1,2-Dilauroyl-sn-glycerol (5.0 g, 10.96 mmol) and tetrazole (29.2 mL of 0.45 M sol in acetonitrile, 13.15 mmol) in 40 mL anhydrous CH₂Cl₂, dibenzyl diisopropyl phosphoramidite (4.54 g, 13.15 mmol) was added and stirred at room temperature for 2 h. The contents were diluted with 100 mL of CH₂Cl₂ and then washed with 5% aqueous NaHCO₃ (2×50 mL), brine (2×50 mL), dried over Na₂SO₄, concentrated in vacuo and the oily residue (7.68 g) was dried in a desiccator for 8 h and used as such in the next reaction. A solution of this phosphite, 2-benzyloxy-1,3-propanediol (0.8 g, 4.38 mmol), pyridine (4.43 mL, 54.77 mmol) and Et₃N (7.63 mL, 54.77 mmol) in CH₂Cl₂ (40 mL) was cooled to −40° C. and pyridinium tribromide (5.25 g, 16.42 mmol) was added at a time. The mixture was stirred at the same temperature for 1 h and gradually allowed to attain room temperature over a period of 2 h and treated with water (30 mL). The contents were diluted with EtOAc (150 mL) and the organic layer was washed successively with aqueous 5% NaHCO₃ (2×50 mL), water (50 mL) and brine (50 mL), dried (Na₂SO₄) and concentrated. The residue was purified on SiO₂ column (8% acetone in CH₂Cl₂) to give 3.8 g (62%) of the product as colorless syrup. TLC (SiO₂) hexane/EtOAc (3:2) Rf˜0.44.

### 2B. 1,3-bis [(1,2-dilauroyl-sn-glycero-3)-phosphoryl]glycerol diammonium salt (Tetralauroyl cardiolipin)

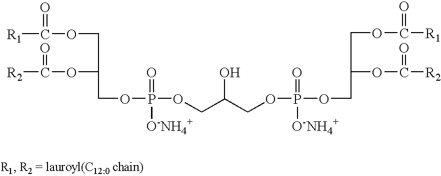

A solution of protected cardiolipin from 2A (1.5 g, 1.07 mmol) in tetrahydrofuran (25 mL) was hydrogenated at 50 psi over 10% Pd/C (600 mg) for 10 h. The catalyst was filtered off over celite bed, treated with 2 mL of 30% ammonia solution and concentrated, the residue was dissolved in CHCl₃, filtered through a 0.25μ filter and precipitated with acetone to give tetralauroyl (C_{12:0}) cardiolipin (1.0 g, 80%) as a white solid. TLC (SiO₂) CHCl₃/MeOH/NH₄OH (6.5:2.5:0.5) R_{f}˜0.48. ¹H NMR δ (CDCl₃, 500 MHz) 0.88 (t, J=7.0 Hz, 12H), 1.22-1.34 (br s, 64H), 1.52-1.66 (m, 8H), 2.26-2.34 (m, 8H), 2.94 (bs, 1H), 3.82-3.98 (m, 9H), 4.12-4.18 (m, 2H), 4.35-4.42 (m, 2H), 5.14-5.24 (m, 2H), 7.41 (bs, 8H). ¹³C NMR δ (CDCl₃, 125 MHz) 14.07, 22.67, 24.87, 24.93, 29.18, 29.22, 29.36, 29.37, 29.40, 29.57, 29.60, 29.66, 29.67, 29.69, 29.72, 31.91, 34.09, 34.28, 62.62, 63.57, 66.77, 69.47, 70.29, 173.25, 173.56. FTIR (ATR) 3207, 3035, 2956, 2918, 2850, 1737, 1467, 1378, 1206, 1092, 1067 cm⁻¹. ESI-MS (negative), m/z 1150 (M−2NH₄⁺+Na⁺), 1127.4 (M−2NH₄⁺), 1128.4 (M−2NH₄⁺+H⁺), 928.4 (M−2NH₄⁺—RCOO⁻), 563.7 (M−2NH₄⁺)²⁻. Anal. Calculated for C₅₇H₁₁₆N₂O₁₇P₂: C, 58.84; H, 10.05; N, 2.41: P, 5.32. Found: C, 57.75; H, 9.83; N, 2.34: P, 5.28.

### EXAMPLE 3

### Synthesis of Tetralauroyl Cardiolipin

In this method the tetralauroyl cardiolipin was synthesized by 2-cyanoethyl phosphoramidite.

### 3A. 2-Benzyl-1,3-bis [(1,2-dilauroyl-sn-glycero-3)-phosphoryl]glycerol dicyanoethyl ester

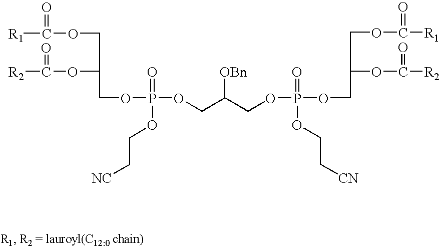

To a mixture of 1,2-dilauroyl-sn-glycerol (1.74 g, 3.79 mmol) and N,N-diisopropylethylamine (545 mg, 4.22 mmol) in anhydrous ether (20 mL) under argon atmosphere was added 2-cyanoethyl diisopropylchlorophosphoramidite (1 g, 4.22 mmol). The mixture was stirred at room temperature for 1 h, the separated disiopropylamine hydrochloride was filtered, and the filtrate was concentrated in vacuo. The residue was as such used for the phosphorylation.

To a mixture of above phosphoramidite and 1H-tetrazole (9.4 mL of 0.45 M sol in acetonotrile, 4.22 mmol) in anhydrous CH₂Cl₂ (30 mL) was added a solution of 2-benzyloxy 1,3-propanediol (312 mg, 1.71 mmol) in CH₂Cl₂ (5 mL). The reaction mixture was stirred at room temperature for 3 h and cooled to −40° C. and m-CPBA (1.36 g, 4.73 mmol was added in portions. After stirring at −40° C. for 30 minutes, the reaction mixture was warmed to room temperature, diluted with CH₂Cl₂ (200 mL), washed {saturated aq Na₂SO₃ (2×50 mL), saturated aq NaHCO₃ (2×50 mL), brine (2×50 mL)} dried (Na₂SO₄) and concentrated. The residue was purified on SiO₂ column (1:4 acetone:CH₂Cl₂) to give 1.48 g (70%) as colorless syrup. TLC (SiO₂) EtOAc/CH₂Cl₂ (1:3) R_{f}˜0.48. ¹H NMR δ (CDCl₃, 500 MHz) 0.88 (t, J=7.0 Hz, 12H), 1.22-1.39 (m, 64H), 1.53-1.66 (m, 8H), 2.26-2.36 (m, 8H), 2.64-2.75 (m, 4H), 3.83-3.88 (m, 1H), 4.06-4.36 (m, 16H), 4.67 (s, 2H), 5.19-5.28 (m, 2H), 7.28-7.38 (m, 5H).

### 3B. 1,3-bis [(1,2-dilauroyl-sn-glycero-3)-phosphoryl]-2-benzylglycerol diammonium salt

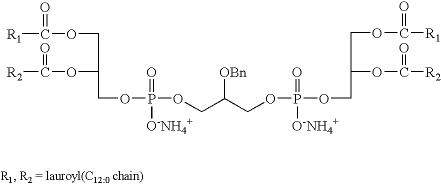

A solution of the precursor from 3A (1.48 g, 1.2 mmol) and Et₃N (1.66 mL, 12 mmol) in 10 mL acetonitrile was stirred overnight (TLC showed no remaining starting material) and evaporated to dryness. The residue was converted into ammonium salt by adding 2 mL of NH₄OH and purified on SiO₂ column (15% MeOH in CH₂Cl₂ containing 1% NH₄OH) to give 850 mg (60%) as colorless syrup that slowly solidified. ¹H NMR δ (CDCl₃, 500 MHz) 0.88 (t, J=7.0 Hz, 12H), 1.22-1.39 (m, 64H), 1.53-1.63 (m, 8H), 2.22-2.34 (m, 8H), 3.66-3.76 (m, 1H), 3.82-4.06 (m, 8H), 4.08-4.18 (m, 2H), 4.26-4.37 (m, 2H), 4.60 (s, 2H), 5.14-5.26 (m, 2H), 7.22-7.36 (m, 5H), 7.49 (bs, 8H).

### 3C. 1,3-bis [(1,2-dilauroyl-sn-glycero-3)-phosphoryl]glycerol diammonium salt (Tetralauroyl cardiolipin)

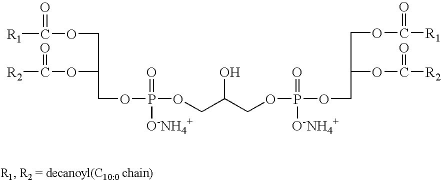

A solution of protected cardiolipin from 3B (1.12 g, 0.89 mmol) in tetrahydrofuran (25 mL) was hydrogenated at 50 psi over 10% Pd/C (450 mg) for 10 h. The catalyst was filtered off over celite bed and concentrated, the residue was dissolved in CHCl₃, filtered through a 0.25μ filter and precipitated with acetone to give tetralauroyl (C_{12:0}) cardiolipin (750 mg, 75%) cardiolipin as a white solid. TLC (SiO₂) column CHCl₃/MeOH/NH₄OH (6.5:2.5:0.5) R_{f}˜0.48. The tetralauroyl cardiolipin prepared by this method described herein is identical to that of 2B in all aspects.

### EXAMPLE 4

### Synthesis of Tetradecanoyl Cardiolipin

### 4A. Synthesis of 1,2-Didecanoyl-3-benzyl-sn-glycerol

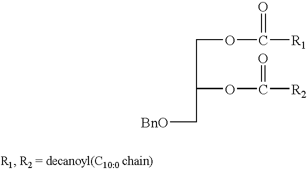

To an ice cooled solution of (R)-(+)-3-benzyloxy-1,2-propanediol (2.0 g, 10.97 mmol) and Et₃N (6.89 mL, 49.36 mmol) in CH₂Cl₂ (30 mL) was added decanoyl chloride (5.1 mL, 24.69 mmol) dropwise over a period of 10 minutes followed by 4-(Dimethylamino)pyridine (DMAP, 268 mg, 2.19 mmol). The reaction mixture was stirred at room temperature for 12 h, diluted with CH₂Cl₂ (200 mL) washed successively with water (100 mL) and brine (100 mL), dried (Na₂SO₄) and concentrated. The residue was purified on SiO₂ column (3% EtOAc in hexane) to give 4.5 g (83%) of the product as colorless syrup. TLC (SiO₂) hexane/EtOAc (9:1) R_{f}˜0.54. ¹H NMR δ (CDCl₃, 300 MHz) 0.87 (t, J=7.0 Hz, 6H), 1.22-1.34 (m, 24H), 1.52-1.66 (m, 4H), 2.22-2.34 (m, 4H), 3.58 (d, J=4.2 Hz, 2H), 4.18 (dd, J=6.4 and 11.9 Hz, 1H), 4.34 (dd, J=6.4 and 11.9 Hz, 1H), 4.51 (d, J=12.2 Hz, 1H), 4.57 (d, J=12.2 Hz, 1H), 5.21-5.28 (m, 1H), 7.28-7.36 (m, 5H).

### 4B. 1,2-Didecanoyl-sn-glycerol

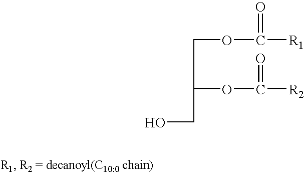

A solution of protected didecanoylglycerol from 4A (4.68 g, 9.55 mmol) in EtOH:EtOAc:AcOH (9:1:0.1) (40 mL) was hydrogenated at 40 psi over 10% Pd/C (600 mg, 10%) for 3 h. The catalyst was filtered off over celite bed and concentrated; the resulting DDG (3.52 g, 92%) was dried under high vacuum. TLC (SiO₂) hexane/EtOAc (3:2) R_{f}˜0.39. ¹H NMR δ (CDCl₃, 300 MHz) 0.87 (t, J=7.0 Hz, 6H), 1.22-1.34 (m, 24H), 1.52-1.66 (m, 4H), 2.03 (t, J=6.2 Hz, 1H, D₂O exchangeable), 2.32 (t, J=7.6 Hz, 2H), 2.35 (t, J=7.6 Hz, 2H) 3.73 (t, J=6.0 Hz, 2H)), 4.22 (dd, J=5.8 and 11.9 Hz, 1H), 4.33 (dd, J=5.8 and 11.9 Hz, 1H), 5.08 (quintet, J=5.1 Hz, 1H).

### 4C. 2-Benzyl-1,3-bis [(1,2-didecanoyl-sn-glycero-3)-phosphoryl]glycerol dibenzyl ester

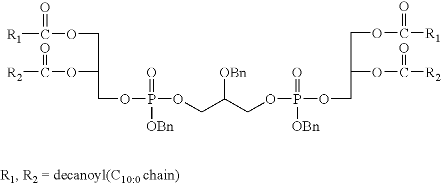

A solution of 1,2-didecanoyl-sn-glycerol from 4B (3.52 g, 8.8 mmol), benzyl N, N-tetraisopropyl phosphoramidite (3.26 g, 9.68 mmol) and 1H-tetrazole (21.51 mL of 0.45 M sol in acetonitrile, 9.68 mmol) in CH₂Cl₂ (25 mL) was stirred at room temperature under argon for 3 h. A solution of 2-benzyloxy 1,3-propanediol (712 mg, 3.9 mmol) in CH₂Cl₂ (10 mL) was added followed by 1H-tetrazole (21.51 mL of 0.45 M sol in acetonitrile, 9.68 mmol) and stirred for 3 h. The reaction mixture was cooled to −40° C. and m-CPBA (5.06 g, 17.6 mmol) was added in portions. After stirring at −40° C. for 30 minutes, the reaction mixture was warmed to room temperature, diluted with CH₂Cl₂ (200 mL), washed {saturated aq Na₂SO₃ (2×50 mL), saturated aq NaHCO₃ (2×50 mL), brine (2×50 mL)} dried (Na₂SO₄) and concentrated. The residue was purified on SiO₂ column (2:3 EtOAc:hexane) to give 3.21 g (64%) of protected cardiolipin as colorless syrup. TLC (SiO₂) hexane/EtOAc (3:2) R_{f}˜0.44. ¹H NMR δ (CDCl₃, 500 MHz) 0.88 (t, J=7.0 Hz, 12H), 1.22-1.34 (m, 48H), 1.52-1.66 (m, 8H), 2.22-2.29 (m, 8H), 3.70-3.78 (m, 1H), 4.01-4.16 (m, 10H), 4.22-4.28 (m, 2H), 4.60 (d, J=7.8 Hz, 2H), 5.01-5.05 (m, 4H), 5.15-5.18 (m, 2H), 7.28-7.36 (m, 15H).

### 4D. 1,3-bis [(1,2-didecanoyl-sn-glycero-3)-phosphoryl]glycerol diammonium salt

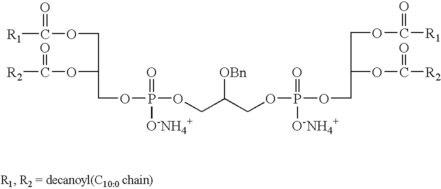

A solution of protected cardiolipin from 4C (1.5 g, 1.16 mmol) in tetrahydrofuran (25 mL) was hydrogenated at 50 psi over 10% Pd/C (600 mg) for 10 h. The catalyst was filtered off over celite bed, treated with 2 mL of 30% ammonia solution and concentrated. The residue was dissolved in CHCl₃, filtered through a 0.25μ filter and concentrated to give tetradecanoyl (C_{10:0}) cardiolipin (1.0 g, 80%) as a semi solid. TLC (SiO₂) CHCl₃/MeOH/NH₄OH (6.5:2.5:0.5) R_{f}˜0.42. ¹H NMR δ (CDCl₃, 300 MHz) 0.88 (t, J=7.0 Hz, 12H), 1.22-1.34 (br s, 48H), 1.52-1.66 (m, 8H), 2.26-2.34 (m, 8H), 3.62 (bs, 1H), 3.82-3.98 (m, 9H), 4.12-4.18 (m, 2H), 4.35-4.42 (m, 2H), 5.14-5.24 (m, 2H), 7.41 (bs, 8H). ESI-MS (negative), m/z 1038 (M−2NH₄⁺+Na⁺), 1015 (M−2NH₄⁺), 843.7 (M−2NH₄⁺—RCOO⁻), 507.5 (M−2NH₄⁺)²⁻.

### EXAMPLE 5

### Synthesis of Tetraoctanoyl Cardiolipin

### 5A. 1,2-Dioctanoyl-3-benzyl-sn-glycerol

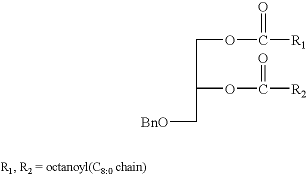

To a solution of (R)-(+)-3-benzyloxy-1,2-propanediol (4.0 g, 21.95 mmol) in anhydrous pyridine (40 mL) was added octanoyl chloride (8.93 g, 54.87 mmol) dropwise over a period of 10 minutes followed by DMAP (267 mg, 2.19 mmol). The reaction mixture was stirred at 55° C. for 48 h, diluted with EtOAc (300 mL) washed successively with water (100 mL), 1N HCl (2×100 mL) and brine (100 mL), dried (Na₂SO₄) and concentrated. The residue was purified on SiO₂ column (3% EtOAc in hexane) to give 7.3 g (75%) of the product as colorless syrup. TLC (SiO₂) hexane/EtOAc (9:1) R_{f}˜0.52. ¹H NMR δ (CDCl₃, 500 MHz) 0.87 (t, J=7.0 Hz, 6H), 1.22-1.34 (m, 16H), 1.52-1.66 (m, 4H), 2.22-2.34 (m, 4H), 3.58 (d, J=4.2 Hz, 2H), 4.18 (dd, J=6.4 and 11.9 Hz, 1H), 4.34 (dd, J=6.4 and 11.9 Hz, 1H), 4.51 (d, J=12.2 Hz, 1H), 4.57 (d, J=12.2 Hz, 1H), 5.21-5.28 (m, 1H), 7.28-7.36 (m, 5H).

### 5B. 12-Dioctanoyl-sn-glycerol

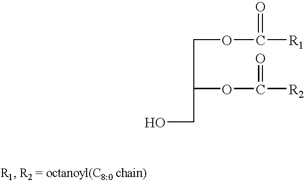

A solution of protected dioctanoyl glycerol from 5A (6.8 g, 15.66 mmol) in EtOH:EtOAc:AcOH (9:1:0.1) (30 mL) was hydrogenated at 40 psi over 10% Pd/C (900 mg, 10%) for 3 h. The catalyst was filtered off over celite bed and concentrated; the resulting DOG (5.0, 93%) was dried under high vacuum. TLC (SiO₂) hexane/EtOAc (3:2) R_{f}˜0.31. ¹H NMR δ (CDCl₃, 300 MHz) 0.87 (t, J=7.0 Hz, 6H), 1.22-1.34 (m, 16H), 1.52-1.66 (m, 4H), 2.03 (t, J=6.2 Hz, 1H, D₂O exchangeable), 2.32 (t, J=7.6 Hz, 2H), 2.35 (t, J=7.6 Hz, 2H) 3.73 (t, J=6.0 Hz, 2H)), 4.22 (dd, J=5.8 and 11.9 Hz, 1H), 4.33 (dd, J=5.8 and 11.9 Hz, 1H), 5.08 (quintet, J=5.1 Hz, 1H).

### 5C. 2-Benzyl-1,3-bis [(1,2-dioctanoyl-sn-glycero-3)-phosphoryl]glycerol dibenzylester

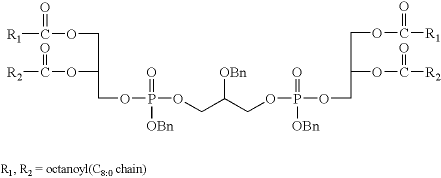

To a solution of 1,2-Dioctanoyl-sn-glycerol from 5B (5.0 g, 14.53 mmol) and tetrazole (40.3 mL of 0.45 M sol in acetonitrile, 18.16 mmol) in 50 mL anhydrous CH₂Cl₂, dibenzyl diisopropyl phosphoramidite (6.26 g, 18.16 mmol) was added and stirred at room temperature for 2 h. The contents were diluted with 200 mL of EtOAc and then washed with 5% aqueous NaHCO₃ (2×50 mL), brine (2×50 mL), dried over Na₂SO₄, concentrated in vacuo and the oily residue (7.0 g) was dried in a desiccator for 8 h and used as such in the next reaction.

A solution of above phosphite, 2-benzyloxy-1,3-propanediol (0.660 g, 3.63 mmol), pyridine (10.6 mL, 131.13 mmol) and Et₃N (8.0 mL, 65.65 mmol) in CH₂Cl₂ (40 mL) was cooled to −40° C. and pyridinium tribromide (6.3 g, 19.69 mmol) was added at a time. The mixture was stirred at the same temperature for 1 h and gradually allowed to attain room temperature over a period of 2 h and treated with water (30 mL). The contents were diluted with EtOAc (250 mL) and the organic layer was washed successively with aqueous 5% NaHCO₃ (2×50 mL), water (50 mL) and brine (50 mL), dried (Na₂SO₄) and concentrated. The residue was purified on SiO₂ column (10% acetone in CH₂Cl₂) to give 2.72 g (64%) of the product as colorless syrup. TLC (SiO₂) hexane/EtOAc (3:2) R_{f}˜0.44. ¹H NMR δ (CDCl₃, 500 MHz) 0.87 (t, J=7.0 Hz, 6H), 0.89 (t, J=7.0 Hz, 6H), 1.22-1.34 (m, 32H), 1.52-1.62 (m, 8H), 2.22-2.29 (m, 8H), 3.70-3.78 (m, 1H), 4.01-4.16 (m, 10H), 4.22-4.28 (m, 2H), 4.60 (d, J=7.8 Hz, 2H), 5.01-5.05 (m, 4H), 5.15-5.18 (m, 2H), 7.28-7.36 (m, 15H).

### 5D. 1,3-bis [(1,2-dioctanoyl-sn-glycero-3)-phosphoryl]-2-benzyl glycerol diammonium salt

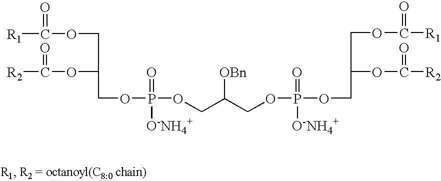

A solution of protected cardiolipin from 5C (2.5 g, 2.12 mmol) in 2-butanone (15 mL) and sodium iodide (956 mg, 6.36 mmol) was refluxed at 90° C. for 3 h. The volatiles were evaporated and the residue was purified on SiO₂ column (20% methanol in CH₂Cl₂ containing 1% of ammonia) to give 1.52 g (75%) of the product as colorless semisolid. TLC (SiO₂) CHCl₃/MeOH/NH₄OH (6.5:2.5:0.5) R_{f}˜0.53. ¹H NMR δ (CDCl₃, 300 MHz) 0.88 (t, J=7.0 Hz, 12H), 1.22-1.34 (m, 32H), 1.56-1.64 (m, 8H), 2.22-2.34 (m, 8H), 3.66-3.76 (m, 1H), 3.82-4.06 (m, 8H), 4.08-4.18 (m, 2H), 4.26-4.37 (m, 2H), 4.60 (s, 2H), 5.14-5.26 (m, 2H), 7.22-7.36 (m, 5H), 7.49 (bs, 8H).

### 5E. 1,3-bis [(1,2-dioctanoyl-sn-glycero-3)-phosphoryl]glycerol diammonium salt

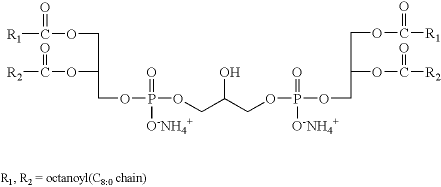

A solution of protected cardiolipin from 5D (1.5 g, 1.27 mmol) in tetrahydrofuran (25 mL) was hydrogenated at 50 psi over 10% Pd/C (600 mg) for 10 h. The catalyst was filtered off over celite bed, treated with 2 mL of 30% ammonia solution and concentrated. The residue was dissolved in CHCl₃, filtered through a 0.25μ filter and concentrated to give C₈ cardiolipin (950 mg, 80%) as a semi solid. TLC (SiO₂) CHCl₃/MeOH/NH₄OH (6.5:2.5:0.5) R_{f}˜0.43. ¹H NMR δ (CDCl₃, 500 MHz) 0.88 (t, J=7.0 Hz, 12H), 1.22-1.34 (m, 32H), 1.56-1.64 (m, 8H), 2.22-2.34 (m, 8H), 3.82-3.98 (m, 9H), 4.12-4.18 (m, 2H), 4.35-4.42 (m, 2H), 5.14-5.24 (m, 2H), 7.41 (bs, 8H). ESI-MS (negative), m/z 925.7 (M−2NH₄⁺+Na⁺), 903.1 (M−2NH₄⁺), 760.6 (M−2NH₄⁺—RCOO⁻), 451.2 (M−2NH₄+)²⁻.

### EXAMPLE 6

### Synthesis of Tetrahexanoyl Cardiolipin

### 6A. 1,2-Dihexanoyl-3-benzyl-sn-glycerol

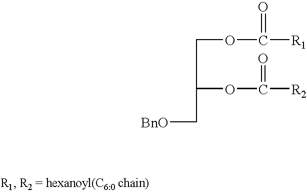

To a solution of (R)-(+)-3-benzyloxy-1,2-propanediol (2.6 g, 14.26 mmol) in anhydrous pyridine (30 mL) was added hexanoyl chloride (4.8 g, 35.67 mmol) dropwise over a period of 10 minutes followed by DMAP (175 mg, 1.42 mmol). The reaction mixture was stirred at 55° C. for 48 h, diluted with EtOAc (200 mL) washed successively with water (100 mL), 1N HCl (2×100 mL) and brine (100 mL), dried (Na₂SO₄) and concentrated. The residue was purified on SiO₂ column (3% EtOAc in hexane) to give 4.1 g (76%) of the product as colorless syrup. TLC (SiO₂) hexane/EtOAc (9:1) R_{f}˜0.48. ¹H NMR δ (CDCl₃, 500 MHz) 0.87 (t, J=7.0 Hz, 6H), 1.22-1.34 (m, 8H), 1.52-1.66 (m, 4H), 2.22-2.34 (m, 4H), 3.58 (d, J=4.2 Hz, 2H), 4.18 (dd, J=6.4 and 11.9 Hz, 1H), 4.34 (dd, J=6.4 and 11.9 Hz, 1H), 4.51 (d, J=12.2 Hz, 1H), 4.57 (d, J=12.2 Hz, 1H), 5.21-5.28 (m, 1H), 7.28-7.36 (m, 5H).

### 6B. 1,2-Dihexanoyl-sn-glycerol

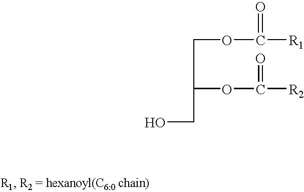

A solution of protected dihexanoylglycerol from 6A (3.1 g, 8.2 mmol) in EtOH:EtOAc:AcOH (9:1:0.1) (30 mL) was hydrogenated at 40 psi over 10% Pd/C (600 mg, 10%) for 3 h. The catalyst was filtered off over celite bed and concentrated; the resulting glycerol (3.52 g, 92%) was dried under high vacuum. TLC (SiO₂) hexane/EtOAc (3:2) R_{f}˜0.34. ¹H NMR δ (CDCl₃, 300 MHz) 0.87 (t, J=7.0 Hz, 6H), 1.22-1.34 (m, 8H), 1.52-1.66 (m, 4H), 2.03 (t, J=6.2 Hz, 1H, D₂O exchangeable), 2.32 (t, J=7.6 Hz, 2H), 2.35 (t, J=7.6 Hz, 2H) 3.73 (t, J=6.0 Hz, 2H)), 4.22 (dd, J=5.8 and 11.9 Hz, 1H), 4.33 (dd, J=5.8 and 11.9 Hz, 1H), 5.08 (quintet, J=5.1 Hz, 1H).

### 6C. 2-Benzyl-1,3-bis [(1,2-dihexanoyl-sn-glycero-3)-phosphoryl]glycerol dibenzyl ester

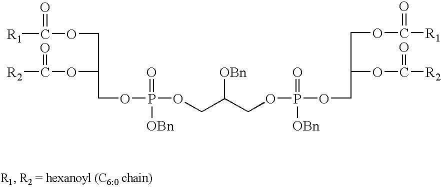

To a solution of 1,2-Dihexanoyl-sn-glycerol from 6B (3.5 g, 13.19 mmol) and tetrazole (35.1 mL of 0.45 M sol in acetonitrile, 15.83 mmol) in 40 mL anhydrous CH₂Cl₂, dibenzyl diisopropyl phosphoramidite (5.46 g, 15.83 mmol) was added and stirred at room temperature for 2 h. The contents were diluted with 200 mL of CH₂Cl₂ and then washed with 5% aqueous NaHCO₃ (2×50 mL), brine (2×50 mL), dried over Na₂SO₄, concentrated in vacuo and the oily residue (7.0 g) was dried in a desiccator for 8 h and used as such in the next reaction.

A solution of above phosphite, 2-benzyloxy-1,3-propanediol (0.957 g, 5.25 mmol), pyridine (10.6 mL, 131.13 mmol) and Et₃N (8.0 mL, 65.65 mmol) in CH₂Cl₂ (40 mL) was cooled to −40° C. and pyridinium tribromide (6.3 g, 19.69 mmol) was added at a time. The mixture was stirred at the same temperature for 1 h and gradually allowed to attain room temperature over a period of 2 h and treated with water (30 mL). The contents were diluted with EtOAc (250 mL) and the organic layer was washed successively with aqueous 5% NaHCO₃ (2×50 mL), water (50 mL) and brine (50 mL), dried (Na₂SO₄) and concentrated. The residue was purified on SiO₂ column (10% acetone in CH₂Cl₂) to give 3.57 g (64%) of the product as colorless syrup. TLC (SiO₂) hexane/EtOAc (3:2) R_{f}˜0.40. ¹H NMR δ (CDCl₃, 500 MHz) 0.87 (t, J=7.0 Hz, 6H), 0.89 (t, J=7.0 Hz, 6H), 1.22-1.34 (m, 16H), 1.52-1.62 (m, 8H), 2.22-2.29 (m, 8H), 3.70-3.78 (m, 1H), 4.01-4.16 (m, 10H), 4.22-4.28 (m, 2H), 4.60 (d, J=7.8 Hz, 2H), 5.01-5.05 (m, 4H), 5.15-5.18 (m, 2H), 7.28-7.36 (m, 15H).

### 6D. 1,3-bis [(1,2-dihexanoyl-sn-glycero-3)-phosphoryl] glycerol diammonium salt

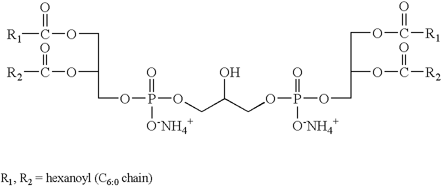

A solution of protected cardiolipin from 6C (1.5 g, 1.41 mmol) in tetrahydrofuran (25 mL) was hydrogenated at 50 psi over 10% Pd/C (600 mg) for 10 h. The catalyst was filtered off over celite bed, treated with 2 mL of 30% ammonia solution and concentrated. The residue was dissolved in CHCl₃, filtered through a 0.25μ filter and concentrated to give C₆ cardiolipin (940 mg, 81%) as a semi solid. TLC (SiO₂) CHCl₃/MeOH/NH₄OH (6.5:2.5:0.5) R_{f}˜0.36. ¹H NMR δ (CDCl₃, 500 MHz) 0.88 (t, J=7.0 Hz, 12H), 1.22-1.34 (m, 16H), 1.56-1.64 (m, 8H), 2.22-2.34 (m, 8H), 3.82-3.98 (m, 9H), 4.12-4.18 (m, 2H), 4.35-4.42 (m, 2H), 5.14-5.24 (m, 2H), 7.41 (bs, 8H). ESI-MS (negative), m/z 813 (M−2NH₄⁺+Na⁺), 791 (M−2NH₄⁺+H⁺), 395 (M−2NH₄⁺)²⁻.

### EXAMPLE 7

### Synthesis of Tetraoleoyl Cardiolipin (Unsaturated)

### 7A. Cis-5-Levulinoyl-2-phenyl-1,3-dioxane

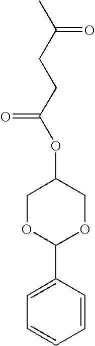

To a solution of cis-1,3-O-benzylideneglycerol (5.0 g, 27.74 mmol) in CH₂Cl₂ (80 mL) at 0° C., DCC (8.58 g, 41.61 mmol), DMAP (3.38 g, 27.74) and levulinic acid (4.0 g, 34.68 mmol) were sequentially added. The mixture was stirred for 24 hrs at room temperature. The separated urea was filtered, the filtrate was concentrated and purified by flash chromatography with 80% EtOAc/hexane as eluent to afford the product as a white solid (5.86 g, 76%). TLC (SiO₂) hexane/EtOAc (1:1) R_{f}˜0.51. ¹H NMR δ (CDCl₃, 300 MHz) 2.19 (s, 3H), 2.58-2.89 (m, 4H), 4.18 (dd, J=12.0, 1.5 Hz, 2H), 4.25 (dd, J=12.0, 1.5 Hz, 2H), 4.71 (dd, J=1.5, 1.5 Hz, 1H), 5.55 (s, 1H), 7.24-7.53 (m, 5H).

### 7B. 2-Levulinoyl-1,3-propanediol

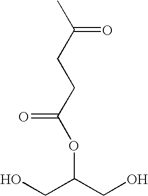

A solution of protected glycerol 7A (5.8 g, 20.86 mmol) in EtOH:EtOAc (1:1) (100 mL) was hydrogenated at 50 psi over 10% Pd/C (800 mg, 10%) for 8 h. The catalyst was filtered off over celite bed and concentrated; the resulting 2-levulinoyl glycerol was purified by flash chromatography with 6% MeOH/CH₂Cl₂ as eluent to afford the product as a white solid (2.82 g, 70%). TLC (SiO₂) MeOH/CH₂Cl₂ (1:9) R_{f}˜0.31. ¹H NMR δ (CDCl₃, 300 MHz) 2.19 (s, 3H), 2.58 (dd, J=6.0, 6.0 Hz, 2H), 2.80 (dd, J=6.0, 6.0 Hz, 2H), 3.09 (bs, 2H), 3.72-3.87 (m, 4H), 4.90 (q, J=5.1 Hz).

### 7C. 2-Levulinoyl-1,3-bis [(1,2-dioleoyl-sn-glycero-3)-phosphoryl]glycerol dibenzyl ester

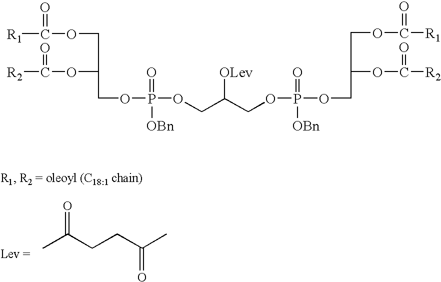

To a solution of 1,2-Dioleoyl-sn-glycerol (1.5 g, 2.41 mmol) and tetrazole (8 mL of 0.45 M sol in acetonitrile, 3.62 mmol) in 20 mL anhydrous CH₂Cl₂, dibenzyl diisopropyl phosphoramidite (1.25 g, 3.62 mmol) was added and stirred at room temperature for 2 h. The contents were diluted with 100 mL of CH₂Cl₂ and then washed with 5% aqueous NaHCO₃ (2×50 mL), brine (2×50 mL), dried over Na₂SO₄, concentrated in vacuo and the oily residue (2.08 g) was dried in a desiccator for 8 h and used as such in the next reaction.

A solution of above phosphite, 2-levulinoyl-1,3-propanediol from 7B (0.185 g, 0.96 mmol), pyridine (2 mL, 24.1 mmol) and Et₃N (1.2 mL, 12.05 mmol) in CH₂Cl₂ (20 mL) was cooled to −40° C. and pyridinium tribromide (1.15 g, 3.61 mmol) was added at a time. The mixture was stirred at the same temperature for 1 h and gradually allowed to attain room temperature over a period of 2 h and treated with water (10 mL). The contents were diluted with EtOAc (100 mL) and the organic layer was washed successively with aqueous 5% NaHCO₃ (2×50 mL), water (50 mL) and brine (50 mL), dried (Na₂SO₄) and concentrated. The residue was purified on SiO₂ column (5% acetone in CH₂Cl₂) to give 1.09 g (66%) of the product as colorless syrup. TLC (SiO₂) hexane/EtOAc (1:2) R_{f}˜0.64. ¹H NMR δ (CDCl₃, 300 MHz) 0.88 (t, J=7.0 Hz, 12H), 1.22-1.34 (m, 80H), 1.52-1.66 (m, 8H), 1.96-2.07 (m, 16H), 2.15 (s, 3H), 2.22-2.31 (m, 8H), 2.52-2.57 (m, 2H), 2.66-2.74 (m, 2H), 4.01-4.32 (m, 12H), 5.01-5.10 (m, 5H), 5.15-5.18 (m, 2H), 5.28-5.39 (m, 8H), 7.28-7.39 (m, 10H).

### 7D. 1,3-bis [(1,2-dioleoyl-sn-glycero-3)-phosphoryl]-2-levulinoyl glycerol diammonium salt

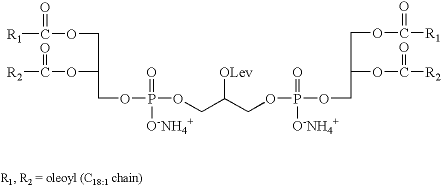

A solution of protected cardiolipin from 7C (0.525 g, 0.324 mmol) in 2-butanone (8 mL) and sodium iodide (145 mg, 0.972 mmol) was refluxed at 90° C. for 3 h. The volatiles were evaporated and the residue was purified on SiO₂ column (10% methanol in CH₂Cl₂ containing 1% of ammonia) to give 240 mg (50%) of the product as colorless semisolid. TLC (SiO₂) CHCl₃/MeOH/NH₄OH (6.5:2.5:0.5) R_{f}˜0.63. ¹H NMR δ (CDCl₃, 300 MHz) 0.88 (t, J=7.0 Hz, 12H), 1.22-1.39 (m, 80H), 1.52-1.65 (m, 8H), 1.96-2.07 (m, 16H), 2.18 (s, 3H), 2.23-2.35 (m, 8H), 2.52-2.59 (m, 2H), 2.71-2.79 (m, 2H), 3.83-4.04 (m, 6H), 4.12-4.23 (m, 4H), 4.31-4.39 (m, 2H), 5.01-5.09 (m, 1H), 5.17-5.26 (m, 2H), 5.28-5.39 (m, 8H), 7.41-7.59 (bs, 8H). ESI-MS (negative), m/z 1576.5 (M−2NH₄⁺+Na⁺), 1554 (M−2NH₄⁺), 1272.2 (M−2NH₄⁺—RCOO⁻), 776 (M−2NH₄⁺)²⁻.

### 7E. 1,3-bis [(1,2-dioleoyl-sn-glycero-3)-phosphoryl]glycerol diammonium salt (Synthesis of Tetraoleoyl Cardiolipin)

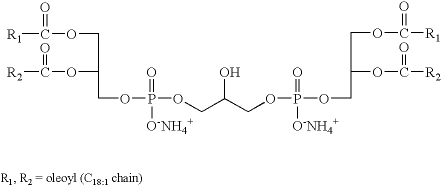

To a solution of lev-protected cardiolipin from 7D (140 mg, 0.088 mmol) in pyridine:acetic acid (3 mL, 4:1) was added hydazine (14 mg, 0.44 mmol) and stirred for 30 minutes. The volatiles were removed in rotavapor and the residue was purified on SiO₂ (10% methanol in CH₂Cl₂ containing 1% of ammonia) to give 80 mg (61%) of the product as colorless semisolid. TLC (SiO₂) (6.5:2.5:0.5) R_{f}˜0.55. ¹H NMR δ (CDCl₃, 500 MHz) 0.88 (t, J=7.0 Hz, 12H), 1.22-1.39 (m, 80H), 1.52-1.65 (m, 8H), 1.82 (bs, 1H), 1.96-2.07 (m, 16H), 2.23-2.35 (m, 8H), 3.83-3.94 (m, 7H), 4.12-4.23 (m, 4H), 4.33-4.39 (m, 2H), 5.17-5.23 (m, 2H), 5.28-5.39 (m, 8H), 7.41-7.59 (bs, 8H). ESI-MS (negative), m/z 1478 (M−2NH₄⁺+Na⁺), 1456 (M−2NH₄⁺), 1174.2 (M−2NH₄⁺—RCOO⁻), 727.5 (M−2NH₄⁺)²⁻.

### Cardiolipin Ether Analogs

### EXAMPLE 8

### Synthesis of Tetralauryl Cardiolipin

### 8A. 2-Benzyl-1,3-bis [(1,2-dilauryl-sn-glycero-3)-phosphoryl]glycerol dibenzyl ester

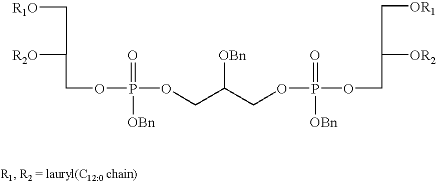

To a stirred solution of 1,2-Dilauryl-sn-glycerol (3.0 g, 7.00 mmol) and tetrazole (19.5 mL of 0.45 M sol in acetonitrile, 8.76 mmol) in 40 mL anhydrous CH₂Cl₂, dibenzyl diisopropyl phosphoramidite (3.02 g, 8.76 mmol) was added and stirred at room temperature for 2 h. The contents were diluted with 100 mL of CH₂Cl₂ and then washed with 5% aqueous NaHCO₃ (2×50 mL), brine (2×50 mL), dried over Na₂SO₄, concentrated in vacuo and the oily residue (4.5 g) was dried in a desiccator for 8 h and used as such in the next step reaction.

A solution of above phosphite, 2-benzyloxy-1,3-propanediol (0.48 g, 2.67 mmol), pyridine (5.43 mL, 66.8 mmol) and Et₃N (4.63 mL, 33.4 mmol) in CH₂Cl₂ (40 mL) was cooled to 40° C. and pyridinium tribromide (3.25 g, 10.02 mmol) was added at a time. The mixture was stirred at the same temperature for 1 h and gradually allowed to attain room temperature over a period of 2 h and treated with water (30 mL). The contents were diluted with EtOAc (150 mL) and the organic layer was washed successively with aqueous 5% NaHCO₃ (2×50 mL), water (50 mL) and brine (50 mL), dried (Na₂SO₄) and concentrated. The residue was purified on SiO₂ column (30% ethylacetate in hexane) to give 2.34 g (67%) of the product as colorless syrup. TLC (SiO₂) hexane/EtOAc (3:2) Rf˜0.47. ¹H NMR δ (CDCl₃, 300 MHz) 0.88 (t, J=6.6 Hz, 12H), 1.22-1.34 (m, 72H), 1.47-1.56 (m, 8H), 3.35-3.58 (m, 14H), 3.72-3.78 (m, 1H), 3.95-4.18 (m, 8H), 4.59-4.61 (m, 2H), 5.02-5.07 (m, 4H), 7.26-7.34 (m, 15H).

### 8B. 1,3-bis [(1,2-dilauryl-sn-glycero-3)-phosphoryl]glycerol diammonium salt (Tetralauryl Cardiolipin)

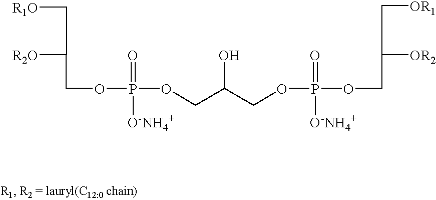

A solution of protected cardiolipin from 8A (650 mg, 0.48 mmol) in tetrahydrofuran (20 mL) was hydrogenated at 50 psi over 10% Pd/C (200 mg) for 6 h. The catalyst was filtered off over celite bed, treated with 2 mL of 30% ammonia solution and concentrated, the residue was dissolved in CHCl₃, filtered through a 0.25μ filter and precipitated with acetone to give C₁₂ cardiolipin (400 mg, 75%) as a white semisolid. TLC (SiO₂) CHCl₃/MeOH/NH₄OH (6.5:2.5:0.5) R_{f}˜0.39. ¹H NMR δ (CDCl₃, 500 MHz) 0.88 (t, J=7.0 Hz, 12H), 1.22-1.34 (m, 72H), 1.52-1.66 (m, 8H), 3.39-3.48 (m, 9H), 3.51-3.61 (m, 6H), 3.80-3.96 (m, 8H), 7.31-7.59 (bs, 8H). ESI-MS (negative), m/z 1094.1 (M-2NH₄⁺+Na⁺), 1072.0 (M−2NH₄⁺+H⁺), 535.5 (M−2NH₄⁺)²⁻.

### EXAMPLE 9

### Synthesis of Tetrahexyl Cardiolipin

### 9A. 2-Benzyl-1,3-bis [(1,2-dihexyl-sn-glycero-3)-phosphoryl]glycerol dibenzyl ester

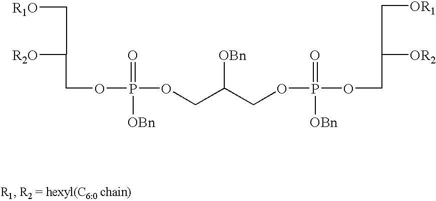

The title compound was prepared according to the method described in example 8A. TLC (SiO₂) hexane/EtOAc (3:2) Rf˜0.39. ¹H NMR δ (CDCl₃, 300 MHz) 0.88 (t, J=6.6 Hz, 12H), 1.22-1.37 (m, 24H), 1.47-1.58 (m, 8H), 3.34-3.58 (m, 14H), 3.72-3.78 (m, 1H), 3.94-4.18 (m, 8H), 4.59-4.61 (m, 2H), 5.02-5.08 (m, 4H), 7.26-7.34 (m, 15H).

### 9B. 1,3-bis [(1,2-dihexyl-sn-glycero-3)-phosphoryl]glycerol diammonium salt (Tetrahexyl Cardiolipin)

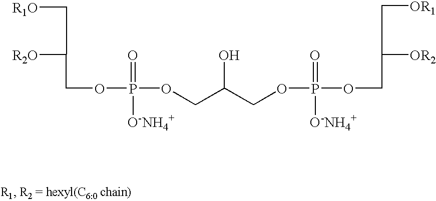

The title compound was prepared according to the method described in example 8B. TLC (SiO₂) CHCl₃/MeOH/NH₄OH (6.5:2.5:0.5) R_{f}˜0.31. ¹H NMR δ (CDCl₃, 500 MHz) 0.88 (t, J=7.0 Hz, 12H), 1.22-1.34 (m, 24H), 1.50-1.61 (m, 8H), 1.88 (bs, 1H), 3.39-3.48 (m, 9H), 3.51-3.61 (m, 6H), 3.80-3.96 (m, 8H), 7.31-7.69 (bs, 8H).

### EXAMPLE 10

This example demonstrates preparation of a cardiolipin-containing liposome composition of the invention. Small unilamellar vesicles are formed by mixing 19.1 μmole of cardiolipin, produced according to the methods described herein, 96.2 μmol of phosphatidyl choline and 64.6 μmol of cholesterol. After thorough stirring, the mixture is evaporated to dryness in a 50 ml round-bottom flask using a rotary evaporator. The subsequent dried lipid film is resuspended in 10 ml sterile non-pyrogenic water. After a 30 minute swelling time, the resulting suspension is sonicated in a fixed temperature bath at 25° C. for 15 minutes. The preparation of liposomes is then lyophilized with trehalose.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments might become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1. Drummond, D. C.; Meyer, O.; Hong, K.; Kirpotin, D. B.; Papahadgopoulos, D. Pharm. Rev., 1999, 51, 691-743.
2. Ramirez, F.; Ioannou, P. V.; Marecek, J. F.; Golding, B. T.; Dodd, G. H. Synthesis. 1976, 11, 769-770.
3. Duralski, A. A.; Spooner, P. J. R.; Watts, A. Tetrahedron Lett. 1989, 30, 3585-3588.
4. Duralski, A. A.; Spooner, P. J. R.; Rankin, S. E.; Watts, A. Tetrahedron Lett. 1998, 39, 1607-1610.
5. Saunders, R. M.; Schwarz. J. Am. Chem. Soc. 1966, 88, 3844-3847.
6. Mishina, I. M.; Vasilenko, A. E.; Stepanov, A. E.; Shvets, V. I. Bioorg. Khim. 1985, 11, 992-994.
7. Stepanov, A. E.; Makarova, I. M.; Shvets, V. I. Zh. Org., Khim. 1984, 20, 985-988.
8. DeHaas, G. H.; Bonsen, P. P. M.; VanDeenen, L. L. M. Biochim. Biophys. Acta, 1966, 116: 114-124.
9. Inoue, K.; Suhara, Y.; Nojima, S. Chem. Pharm. Bull., 1963, 1150-1156.
10. Browne, J. E.; Driver, M. J.; Russel, J. C.; Sammes, P. G. J. Chem. Soc. Perkin Trans. 1. 2000, 653-657.
11. Watanabe, Y.; Inada, E.; Jinno, M.; Ozaki, S. Tetrahedron Lett. 1993, 34, 497-500.
12. Watanabe, Y.; Hirofuji, H.; Ozaki, S. Tetrahedron Lett. 1994, 35, 123-124.
13. Watanabe, Y.; Nakamura, T.; Mitsumoto, H. Tetrahedron Lett. 1997, 38, 7407-7410.
14. Watanabe, Y.; Ishikawa, H. Tetrahedron Lett. 2000, 41, 8509-8512.
15. Watanabe, Y.; Nakatomi, M. Tetrahedron Lett. 1998, 39, 1583-1586.
16. Chen, J.; Feng, L.; Prestwich, G. D. J. Org. Chem. 1998, 63, 6511-6522.
17. Lindberg, J.; Ekeroth, J.; Konradsson, P. J. Org Chem. 2002, 67, 194-199.
18. Chen, J.; Profit, A. A.; Prestwich, G. D. J. Org. Chem. 1996, 61, 6305-6312.
19. Prestwich, G. D. Acc. Chem. Res. 1996, 29, 503-513.
20. Gu, Q. M.; Prestwich, G. D. J. Org Chem. 1996, 61, 8642-8647.
21. Murakami, K.; Molitor, E. J.; Liu, H. W. J. Org. Chem. 1999, 64,648-651.
22. Prestwich, G. D.; Marecek, J. F.; Mourey, R. J.; Thiebert, A. B.; Ferris, C. D.; Danoff, S. K.; Snyder, S. H. J. Am. Chem. Soc. 1991, 113, 1822-1825.
23. Dreef, C. E.; Elie, C. J. J.; Hoogerhout, P.; van der Marel, G. A.; van Boom, J. H. Tetrahedron Lett. 1988, 29, 6513-6516.
24. Inoue, K.; Nojima, S. Chem. Pharm. Bull. 1968, 16, 76-81.
25. Ioannou, P. V.; Marecek, J. F. Chem. Chron. 1986, 15, 205-220.
26. Ramirez, F.; Ioannou, P. V.; Marecek, J. F.; Dodd, G. H.; Golding, B. T. Tetrahedron. 1977, 33, 599-608.
27. Mishina, I. M.; Vasilenko, A. E.; Stepanov, A. E.; Shvets, V. I. Bioorg. Khim. 1987, 13, 1110-1115.
28. Keana, J. F. W.; Shimiju, M.; Jemstedt, K. K. J. Org. Chem. 1986, 51, 2297-2299.
29. Chevallier, J.; Sakai, N.; Robert, F.; Kobayashi, T.; Gruenberg, J.; Matile, S. Org. Lett. 2000, 2, 1859-1861.
30. Wilk, A.; Srinivasachar, K.; Beaucage, S. J. Org. Chem. 1997, 62, 6712-6713.
31. Moriguchi, T.; Yanagi, T.; Kunimori, M.; Wada, T.; Sekine, M. J. Org. Chem. 2000, 65, 8229-8238.

1. A method for preparing a cardiolipin or analogue thereof of formulas I, II or III 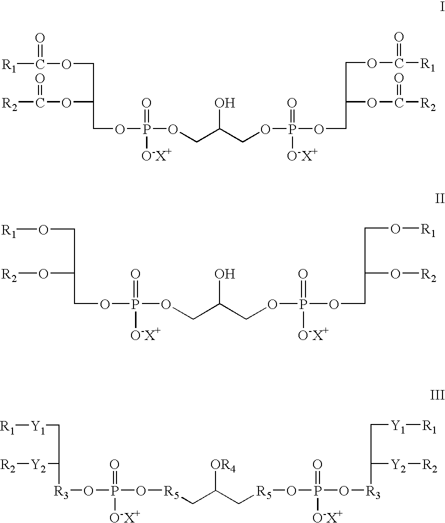
comprising reacting an alcohol of the formula VIII 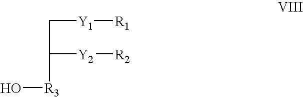
with one or more phosphoramidite reagents and 2-O-protected glycerol or 2-O-subsituted glycerol in the presence of an acid catalyst, wherein, in Formulas I, II, III, or VIII
Y₁ and Y₂ are the same or different and are —O—C(O)—, —O—, —S—, or —NH—C(O)—;
R₁ and R₂ are the same or different and are H, C₂ to C₃₄ saturated or unsaturated alkyl group;
R₃ is (CH₂), and n=0-15;
R₄ is hydrogen, alkyl, substituted alkyl, cycloalkyl, substituted cycloalkyl, a peptide, dipeptide, polypeptide, protein, carbohydrate, heterocyclic, nucleoside or polynucleotide;
R₅ is a linker selected from a group consisting of alkyl, substituted alkyl, cycloalkyl, substituted cycloalkyl, alkyloxy, polyalkyloxy, a peptide, dipeptide, polypeptide, protein and carbohydrate;
X is hydrogen or a non-toxic cation.

2. The method of claim 1, wherein at least one of the phosphoramidite reagents is of formula IV.

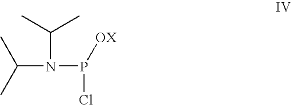

3. The method of claim 1, wherein at least one of the phosphoramidite reagents is of formula V.

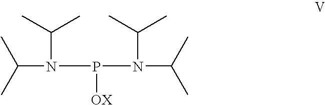

4. A method for preparing cardiolipin or an analogue thereof of formulas I, II, or III; comprising reacting 2-O protected glycerol with one or more phosphotriesters in the presence of pyridinium tribromide.

5. The method of claim 4, wherein one or more of the phosphotriesters are produced by reacting an alcohol of formula VIII with a phosphoramidite reagent of general formula VII.

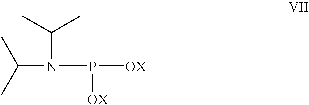

6. The methods of any of claims 2, 3, or 5, wherein X in formulas IV, V, or VII is a phosphate protecting group including alkyl phosphates including ethyl, cyclohexyl, t-butyl; 2-substituted ethyl phosphates including 2-cyanoethyl, 4-cyano-2-butenyl, 2-(methyldiphenylsilyl)ethyl, 2-(trimethylsilyl)ethyl, 2-(triphenylsilyl)ethyl; haloethyl phosphates including 2,2,2-trichloroethyl, 2,2,2-tribromoethyl, 2,2,2-trifluoroethyl; benzyl phosphates including 4-chlorobenzyl, fluorenyl-9-methyl, diphenylmethyl and amidates.

7. The method of claim 1, wherein the acid catalyst is selected from a group consisting of 4,5-dichloroimidazole, 1H-tetrazole, 5-(4-nitrophenyl)-1H-tetrazole, 5-(3,5-dinitrophenyl)-1H-tetrazole, N-methylimidazolium triflate, and N-methylimidazolium perchlorate, 4,5-dicyanoimidazole, 5-ethylthio-1H-tetrazole, and 5-methylthio-1H-tetrazole.

8. The method of claim 1 or 4, wherein the cardiolipin or analogue thereof comprises short-chain fatty acids having between 2 and 14 carbons.

9. The method of claim 8, wherein the cardiolipin or analogue thereof comprises short-chain fatty acids having between 4 and 12 carbons.

10. The method of claim 1 or 4, wherein the cardiolipin or analogue thereof comprises long-chain fatty acids having between 14 and 34 carbons.

11. The method of claim 10, wherein the cardiolipin or analogue thereof comprises long-chain fatty acids having between 14 and 24 carbons.

12. The method of claim 1 or 4, wherein the cardiolipin or analogue thereof is saturated and/or unsaturated.

13. A method for preparing a liposome, comprising preparing a cardiolipin or cardiolipin analogue by the method of claim 1 or 4 and including the cardiolipin or cardiolipin analogue in a liposome.

14. A method for retaining a drug in a liposome, comprising preparing a cardiolipin or cardiolipin analogue by the method of claim 1 or 4 and including the cardiolipin or cardiolipin analogue and a drug in a liposome.

15. A method for retaining drugs in a liposome, comprising preparing a cardiolipin or cardiolipin analogue by the method of claim 1 or 4 and including the cardiolipin or cardiolipin analogue and a mixture of drugs in a liposome.

16. The method of claim 15, wherein the mixture comprises two or more drugs.

17. A composition prepared by the method of claim 1 or 4.

18. The composition of claim 17, which comprises a liposomal composition.

19. The composition of claim 18, further comprising a phosphatidylcholine, a sterol, and a tocopherol.

20. The composition of claim 18, further comprising a phosphatidylcholine selected from a group consisting of dimyristoylphosphatidylcholine, distearoylphosphatidylcholine, dioleylphosphatidylcholine, dipalmitoylphosphatidylcholine, diarachidonoylphosphatidylcholine, egg phosphatidylcholine, soy phosphatidylcholine, hydrogenated soy phosphatidylcholine, and mixtures thereof.

21. The composition of claim 18, further comprising a phosphatidylglycerol selected from a group consisting of dimyristoylphosphatidylglycerol, distearoylphosphatidylglycerol, dioleylphosphatidylglycerol, dipalmitoylphosphatidylglycerol, diarachidonoylphosphatidylglycerol, and mixtures thereof.

22. The composition of claim 18, further comprising a sterol selected from a group consisting of cholesterol, derivatives of cholesterol, coprostanol, cholestanol, cholestane, cholesterol hemisuccinate, cholesterol sulfate, and mixtures thereof.

23. The composition of claim 18, further comprising one or more targeting agents.

24. The composition of claim 18, further comprising a cryoprotectant.

25. The composition of claim 18, further comprising a ligand.

26. The composition of claim 25, wherein the ligand is an antibody or a ligand for a cellular receptor.

27. The composition of claim 18, wherein the composition is in a lyophilized form.

28. The composition of claim 18, further comprising a pharmaceutically acceptable excipient.

29. The composition of claim 17, further comprising one or more active agents.

30. The composition of claim 29, wherein at least one of said active agents is complexed with the cardiolipin or cardiolipin analogue.

31. The composition of claim 18, further comprising one or more active agents.

32. The composition of claim 31, wherein at least one of said active agents is entrapped within the liposomes.

33. A method of delivering an active agent or mixture of active agents to a cell, comprising preparing a composition according to claim 1 or 4 and exposing the composition and one more more active agents to a cell.

34. The method of claim 33, wherein the cell is in vitro.

35. The method of claim 33, wherein the cell is in vivo.

36. A method of treating a human or animal disease, comprising preparing a composition according to claim 1 or 4 and exposing the composition and one or more active agents to a human or animal in need thereof such that the active agent is delivered to the human or animal patient.

37. The method of claim 36, wherein the disease is cancer and at least one of said active agents is an anticancer agent.

38. Use of cardiolipin or a cardiolipin analogue of claim 17 to prepare a medicament to combat aging.

39. Use of cardiolipin or a cardiolipin analogue of claim 17 to prepare a medicament to combat a mammalian disease.

40. The use according to claim 39, wherein said disease is selected from a group consisting of age-related diseases, atherosclerosis, diabetes, heart disease, ischemia, cancer and skin disorders.

41. The use according to claim 38, wherein the cardiolipin is in the form of a liposomal composition.

42. The use according to claim 39, wherein the cardiolipin is in the form of a liposomal composition.

43. A method of treating the effects of aging in a patient, comprising administering the patient a therapeutically effective amount of the cardiolipin or cardiolipin analogue of claim 17 such that the effects of aging are combated in said patient.
